(19) United States
(12) Patent Application Publication
(10) Pub. No.: US 2026/0028415 A1
(43) Pub. Date: Jan. 29, 2026

(54) ANTIBODIES SPECIFICALLY BINDING TO CEACAM5

(71) Applicant: Full-Life Technologies HK Limited, Hong Kong (CN)

(72) Inventors: Chung Lim WONG, Shanghai (CN); Ravi V. J. CHARI, Needham, MA (US); Chieh-Ju LEE, Douliou, Yunlin City (TW); Dian LI, Shanghai (CN)

(21) Appl. No.: 19/143,819

(22) PCT Filed: Dec. 28, 2023
(86) PCT No.: PCT/CN2023/142704

§ 371 Date: Jun. 26, 2025

(30) Foreign Application Priority Data
Dec. 28, 2022 (WO) PCT/CN2022/142838

(51) Int. Cl. C07K 16/30; A61K 35/17; A61K 40/11; A61K 40/31; A61K 40/42; A61K 47/68; C12N 5/0783

(52) U.S. Cl. CPC C07K 16/3007; A61K 35/17; A61K 40/11; A61K 40/31; A61K 40/4266; A61K 47/6853; C12N 5/0636; A61K 2239/13; C07K 2317/24; C07K 2317/31; C07K 2317/565; C07K 2317/569; C07K 2317/77; C12N 2510/00

(57) ABSTRACT

Provided herein are antibodies (e.g., sdAb such as VHH) specifically binding to CEACAM5, constructs and conjugates comprising the antibodies, and methods of making and uses thereof.

### RELATED APPLICATIONS

The present application claims priority of International Application No. PCT/CN2022/142838, filed on Dec. 28, 2022, the disclosure of which is hereby incorporated by reference in its entirety.

### FIELD OF THE APPLICATION

The present application relates to antibodies (e.g., single domain antibodies (sdAbs)) specifically binding to carcinoembryonic antigen-related cell adhesion molecule 5 (CEACAM5). Also provided are antibody constructs and conjugates comprising such anti-CEACAM5 antibodies, methods of making, and uses thereof.

### BACKGROUND OF THE APPLICATION

Carcinoembryonic antigen-related cell adhesion molecule 5 (CEACAM5), also known as CD66e (Cluster of Differentiation 66e) or cancer embryonic antigen (CEA), is a member of the carcinoembryonic antigen (CEA) gene family. CEACAM5 has been found over-expressed in a high percentage of human tumors, including 90% of gastrointestinal, colorectal and pancreatic cancers, 70% of non-small cell lung cancer cells, and 50% of breast cancers. High levels of CEACAM5 have also been implicated with enhanced metastasis and the development of malignancy. Its primary function in the embryonic intestine and colon tumors is adhesion between epithelial cells, and evidence also shows that high CEACAM5 expression is associated with CD133-positive colorectal cancer stem cells, suggesting CEACAM5 as a compelling target for treating malignancies.

### BRIEF SUMMARY OF THE INVENTION

In one aspect, provided herein is an antibody (e.g., sdAb such as VHH) specifically binding to CEACAM5, comprising CDR1, CDR2, and CDR3 of a single domain antibody of anyone selected from the group consisting of SEQ ID NOs: 1-10. In some embodiments, the CDR1, CDR2, and CDR3 are according to IMGT numbering. In some embodiments, the CDR1, CDR2, and CDR3 are according to Kabat numbering.

In some embodiments according to any of the anti-CEACAM5 antibodies described above, the antibody comprises: (1) a CDR1 comprising an amino acid sequence of SEQ ID NO: 11 or an amino acid sequence with one or more (e.g., 1, 2, or 3) amino acid alterations as compared to SEQ ID NO: 11, (2) a CDR2 comprising an amino acid sequence of SEQ ID NO: 21 or an amino acid sequence with one or more (e.g., 1, 2, or 3) amino acid alterations as compared to SEQ ID NO: 21, and (3) a CDR3 comprising an amino acid sequence of SEQ ID NO: 31 or an amino acid sequence with one or more (e.g., 1, 2, or 3) amino acid alterations as compared to SEQ ID NO: 31. In some embodiments, the antibody comprises: (1) a CDR1 comprising an amino acid sequence of SEQ ID NO: 12 or an amino acid sequence with one or more (e.g., 1, 2, or 3) amino acid alterations as compared to SEQ ID NO: 12, (2) a CDR2 comprising an amino acid sequence of SEQ ID NO: 22 or an amino acid sequence with one or more (e.g., 1, 2, or 3) amino acid alterations as compared to SEQ ID NO: 22, and (3) a CDR3 comprising an amino acid sequence of SEQ ID NO: 32 or an amino acid sequence with one or more (e.g., 1, 2, or 3) amino acid alterations as compared to SEQ ID NO: 32. In some embodiments, the antibody comprises: (1) a CDR1 comprising an amino acid sequence of SEQ ID NO: 13 or an amino acid sequence with one or more (e.g., 1, 2, or 3) amino acid alterations as compared to SEQ ID NO: 13, (2) a CDR2 comprising an amino acid sequence of SEQ ID NO: 23 or an amino acid sequence with one or more (e.g., 1, 2, or 3) amino acid alterations as compared to SEQ ID NO: 23, and (3) a CDR3 comprising an amino acid sequence of SEQ ID NO: 33 or an amino acid sequence with one or more (e.g., 1, 2, or 3) amino acid alterations as compared to SEQ ID NO: 33. In some embodiments, the antibody comprises: (1) a CDR1 comprising an amino acid sequence of SEQ ID NO: 14 or an amino acid sequence with one or more (e.g., 1, 2, or 3) amino acid alterations as compared to SEQ ID NO: 14, (2) a CDR2 comprising an amino acid sequence of SEQ ID NO: 24 or an amino acid sequence with one or more (e.g., 1, 2, or 3) amino acid alterations as compared to SEQ ID NO: 24, and (3) a CDR3 comprising an amino acid sequence of SEQ ID NO: 34 or an amino acid sequence with one or more (e.g., 1, 2, or 3) amino acid alterations as compared to SEQ ID NO: 34. In some embodiments, the antibody comprises: (1) a CDR1 comprising an amino acid sequence of SEQ ID NO: 15 or an amino acid sequence with one or more (e.g., 1, 2, or 3) amino acid alterations as compared to SEQ ID NO: 15, (2) a CDR2 comprising an amino acid sequence of SEQ ID NO: 25 or an amino acid sequence with one or more (e.g., 1, 2, or 3) amino acid alterations as compared to SEQ ID NO: 25, and (3) a CDR3 comprising an amino acid sequence of SEQ ID NO: 35 or an amino acid sequence with one or more (e.g., 1, 2, or 3) amino acid alterations as compared to SEQ ID NO: 35. In some embodiments, the antibody comprises: (1) a CDR1 comprising an amino acid sequence of SEQ ID NO: 16 or an amino acid sequence with one or more (e.g., 1, 2, or 3) amino acid alterations as compared to SEQ ID NO: 16, (2) a CDR2 comprising an amino acid sequence of SEQ ID NO: 26 or an amino acid sequence with one or more (e.g., 1, 2, or 3) amino acid alterations as compared to SEQ ID NO: 26, and (3) a CDR3 comprising an amino acid sequence of SEQ ID NO: 36 or an amino acid sequence with one or more (e.g., 1, 2, or 3) amino acid alterations as compared to SEQ ID NO: 36. In some embodiments, the antibody comprises: (1) a CDR1 comprising an amino acid sequence of SEQ ID NO: 17 or an amino acid sequence with one or more (e.g., 1, 2, or 3) amino acid alterations as compared to SEQ ID NO: 17. (2) a CDR2 comprising an amino acid sequence of SEQ ID NO: 27 or an amino acid sequence with one or more (e.g., 1, 2, or 3) amino acid alterations as compared to SEQ ID NO: 27, and (3) a CDR3 comprising an amino acid sequence of SEQ ID NO: 37 or an amino acid sequence with one or more (e.g., 1, 2, or 3) amino acid alterations as compared to SEQ ID NO: 37. In some embodiments, the antibody comprises: (1) a CDR1 comprising an amino acid sequence of SEQ ID NO: 18 or an amino acid sequence with one or more (e.g., 1, 2, or 3) amino acid alterations as compared to SEQ ID NO: 18, (2) a CDR2 comprising an amino acid sequence of SEQ ID NO: 28 or an amino acid sequence with one or more (e.g., 1, 2, or 3) amino acid alterations as compared to SEQ ID NO: 28, and (3) a CDR3 comprising an amino acid sequence of SEQ ID NO: 38 or an amino acid sequence with one or more (e.g., 1, 2, or 3) amino acid alterations as compared to SEQ ID NO: 38. In some embodiments, the antibody comprises: (1) a CDR1 comprising an amino acid sequence of SEQ ID NO: 19 or an amino acid sequence with one or more (e.g., 1, 2, or 3) amino acid alterations as compared to SEQ ID NO: 19, (2) a CDR2 comprising an amino acid sequence of SEQ ID NO: 29 or an amino acid sequence with one or more (e.g., 1, 2, or 3) amino acid alterations as compared to SEQ ID NO: 29, and (3) a CDR3 comprising an amino acid sequence of SEQ ID NO: 39 or an amino acid sequence with one or more (e.g., 1, 2, or 3) amino acid alterations as compared to SEQ ID NO: 39. In some embodiments, the antibody comprises: (1) a CDR1 comprising an amino acid sequence of SEQ ID NO: 20 or an amino acid sequence with one or more (e.g., 1, 2, or 3) amino acid alterations as compared to SEQ ID NO: 20, (2) a CDR2 comprising an amino acid sequence of SEQ ID NO: 30 or an amino acid sequence with one or more (e.g., 1, 2, or 3) amino acid alterations as compared to SEQ ID NO: 30, and (3) a CDR3 comprising an amino acid sequence of SEQ ID NO: 40 or an amino acid sequence with one or more (e.g., 1, 2, or 3) amino acid alterations as compared to SEQ ID NO: 40.

In some embodiments according to any of the anti-CEACAM5 antibodies described above, the antibody comprises an amino acid sequence having at least about 70% identity (e.g., at least about any of 80%, 85%, 90%, 95%, 96%, 97%, 98%, 99%, or more identity) with the amino acid sequence of any of SEQ ID NOs: 1-10 and 71-97. In some embodiments, the antibody comprises an amino acid sequence of any of SEQ ID NOs: 1-10 and 71-97.

In another aspect, provided herein is an antibody construct comprising any of the anti-CEACAM5antibodies described above. In some embodiments, the antibody construct further comprises an Fc fragment, such as an Fc fragment derived from any of IgG1, IgG2, IgG3 and IgG4. In some embodiments, the antibody construct is multi-specific, such as bispecific.

In another aspect, provided herein is nucleic acid encoding any of the anti-CEACAM5 antibodies or antibody constructs described above. In some embodiments, the nucleic acid comprises a polynucleotide sequence having at least about 70% (e.g., at least about any of 80%, 85%, 90%, 95%, 96%, 97%, 98%, 99%, or more) sequence identity with a sequence of any of SEQ ID NOs: 100-109. In some embodiments, the nucleic acid comprises a polynucleotide sequence of anyone selected from the group consisting of SEQ ID NOs: 100-109.

In another aspect, provided herein is a vector comprising any of the nucleic acids as described above.

In another aspect, provided herein is a host cell comprising any of the nucleic acids or the vectors described above.

In another aspect, provided herein is a conjugate comprising: (1) any of the anti-CEACAM5 antibodies or antibody constructs described above, and (2) a drug. In some embodiments, the drug is selected from the group consisting of chemotherapeutic agent, toxin, cytokine, enzyme, immunomodulator, chelator, diagnostic agent, nanoparticle, fluorescent label, and radioisotope. In some embodiments, the conjugate further comprises a linker linking the antibody or antibody construct with the drug.

In another aspect, provided herein is a chimeric antigen receptor (CAR) comprising antibody constructs described above.

In another aspect, provided herein is an engineered T-cell receptor (TCR) comprising antibody constructs described above.

In another aspect, provided herein is a cell (e.g., immune cell, such as effector cell (e.g., effector T cell or NK cell)) expressing any of the CARs or engineered TCRs described above.

In another aspect, provided herein is a pharmaceutical composition, comprising: (1) any of the anti-CEACAM5 antibodies, antibody constructs, nucleic acids, vectors, conjugates, or cells described above, and (2) a pharmaceutically acceptable excipient.

In another aspect, provided herein is a method for treating a disorder in a subject (e.g., human) in need thereof, comprising administrating an effective amount of any of the pharmaceutical compositions described above to the subject. In some embodiments, the disorder is associated with the undesired presence of CEACAM5+ cells. In some embodiments, the disorder is CEACAM5+ cancer.

In another aspect, provided herein is a use of any of the pharmaceutical compositions described above in the manufacture of a medicament for treating a disorder (e.g., CEACAM5+ cancer) in a subject (e.g., human) in need thereof.

In another aspect, provided herein is a method of detecting CEACAM5 in a sample from a subject (e.g., human), comprising contacting the sample with the antibody or the conjugate described above with the sample, wherein detection of the antibody or the conjugate indicates presence of CEACAM5 in the sample.

Also provided are methods of preparing any of the anti-CEACAM5 antibodies or antibody constructs described above, comprising: (a) culturing a host cell comprising any of the isolated nucleic acids or vectors described above, or any of the host cells described above, under a condition effective to express the encoded antibody or antibody construct; and (b) obtaining the expressed antibody or antibody construct from the host cell.

The disclosures of all publications, patents, patent applications and published patent applications referred to herein are hereby incorporated herein by reference in their entirety.

### BRIEF DESCRIPTION OF THE DRAWINGS

FIG. 1 illustrates internalization of exemplary anti-CEACAM5 antibodies in CEACAM5+MKN45 cells.

### DETAILED DESCRIPTION OF THE APPLICATION

Before describing the embodiments in detail, it is to be understood that the present disclosure is not limited to particular compositions or biological systems, which can, of course, vary. It is also to be understood that the terminology used herein is for the purpose of describing particular embodiments only, and is not intended to be limiting.

### Definitions

As used in this specification and the appended claims, the singular forms “a,” “an” and “the” include plural referents unless the content clearly dictates otherwise. Thus, for example, reference to “a molecule” optionally includes a combination of two or more such molecules, and the like.

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, single domain antibodies, so long as they exhibit the desired antigen-binding activity.

A full-length antibody comprises two heavy chains and two light chains. The variable regions of the light and heavy chains are responsible for antigen binding. The variable domains of the heavy chain and light chain may be referred to as “VH” and “VL”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as lgG1 (γ1 heavy chain), lgG2 (γ2 heavy chain), lgG3 (γ3 heavy chain), lgG4 (γ4 heavy chain), lgA1 (α1 heavy chain), or lgA2 (α2 heavy chain).

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (dsdiabody), a single-chain Fv (scFv), an scFv dimer (bivalent diabody), a multispecific antibody formed from a portion of an antibody comprising one or more CDRs, a single domain antibody (e.g., a camelized single domain antibody), a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “single-domain antibody (sdAb, also known as nanobody)” as used herein refers to an antibody fragment consisting of a single monomeric variable antibody domain. Like a whole antibody, it is able to bind selectively to a specific antigen. With a molecular weight of only 12-15 kDa, single-domain antibodies are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain).

The first single-domain antibodies were engineered from heavy-chain antibodies found in camelids; these are called VHH fragments. Cartilaginous fishes also have heavy-chain antibodies (IgNAR, immunoglobulin new antigen receptor), from which single-domain antibodies called VNAR fragments can be obtained. An alternative approach is to split the dimeric variable domains from common immunoglobulin G (IgG) from humans or mice into monomers. Although most research into single-domain antibodies is currently based on heavy chain variable domains, nanobodies derived from light chains have also been shown to bind specifically to target epitopes.

Camelid nanobodies have been shown to be as specific as antibodies, and in some cases they are more robust. They are easily isolated using the same phage panning procedure used for antibodies, allowing them to be cultured in vitro in large concentrations. The smaller size and single domain make these antibodies easier to transform into bacterial cells for bulk production, making them ideal for research purposes.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273:927-948 (1997); MacCallum et al., J. Mol. Biol. 262:732-745 (1996); Abhinandan and Martin, Mol. Immunol., 45:3832-3839 (2008); Lefranc M. P. et al., Dev. Comp. Immunol., 27:55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45:3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR Definitions

| | Kabat¹ | Chothia² | MacCallum³ | IMGT⁴ | AHo⁵ |
|---|---|---|---|---|---|
| VH CDR1 | 31-35 | 26-32 | 30-35 | 27-38 | 25-40 |
| VH CDR2 | 50-65 | 53-55 | 47-58 | 56-65 | 58-77 |
| VH CDR3 | 95-102 | 96-101 | 93-101 | 105-117 | 109-137 |
| VL CDR1 | 24-34 | 26-32 | 30-36 | 27-38 | 25-40 |
| VL CDR2 | 50-56 | 50-52 | 46-55 | 56-65 | 58-77 |
| VL CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Pluckthun, supra

The expression “variable-domain residue-numbering as in Kabat” or “amino-acid position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or hypervariable region (HVR) of the variable domain. For example, a heavy-chain variable domain may include a single amino acid insert (residue 52a according to Kabat) after residue 52 of 112 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

In some embodiments, the CDRs of an antibody can be determined according to the IMGT numbering system. With respect to the IMGT numbering system, (i) the VH CDR1 is typically present at amino acid positions 25 to 35 of the heavy chain; (ii) the VH CDR2 is typically present at amino acid positions 51 to 57 of the heavy chain; and (iii) the VH CDR2 is typically present at amino acid positions 93 to 102 of the heavy chain. With respect to the IMGT numbering system, (i) the VL CDR1 is typically present at amino acid positions 27 to 32 of the light chain; (ii) the VL CDR2 is typically present at amino acid positions 50 to 52 of the light chain; and (iii) the VL CDR3 is typically present at amino acid positions 89 to 97 of the light chain.

“Framework” or “FR” residues are those variable-domain residues other than the CDR residues as herein defined.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32 (5): 1792-1797, 2004; Edgar, R. C., BMC Bioinformatics 5 (1): 113, 2004).

The term “Fc region” or “fragment crystallizable region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen. As used herein, the terms “specifically binds,” “specifically recognizing,” and “is specific for” refer to measurable and reproducible interactions, such as binding between an antibody and an antigen thereof, which is determinative of the presence of the target or antigen in the presence of a heterogeneous population of molecules, including biological molecules. For example, an antibody that specifically recognizes an antigen is the antibody that binds this antigen with greater affinity, avidity, more readily, and/or with greater duration than its bindings to other targets or antigens. In some embodiments, the extent of binding of antibody to an unrelated target or antigen is less than about 10% of the binding of the antibody to the antigen thereof as measured, e.g., by a radioimmunoassay (RIA). In some embodiments, an antibody that specifically binds the antigen thereof has a dissociation constant (K_{D}) of ≤10⁵ M, ≤10⁶ M, ≤10⁻⁷ M, ≤10⁻⁸ M, ≤10⁹ M, ≤10⁻¹⁰ M, ≤10⁻¹¹ M, or ≤10⁻¹² M. In some embodiments, said specific binding can include, but does not require exclusive binding. Binding specificity of the antibody can be determined experimentally by methods known in the art. Such methods comprise, but are not limited to, e.g., Western blots, ELISA-, RIA-, ECL-, IRMA-, EIA-, BIACORE™-tests and peptide scans.

An “isolated” nucleic acid molecule encoding a polypeptide, antibody or antibody construct as described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. In some embodiments, the isolated nucleic acid molecules encoding the antibody or antibody construct described herein is in a form other than in the form or setting in which it is found in nature.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, and may contain mutations. Mutant progeny that has the same function or biological activity as screened or selected for in the originally transformed cell are included herein.

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the application contemplate any one or more of these aspects of treatment.

In the context of cancer, the term “treating” includes any or all of: inhibiting growth of cancer cells, reducing tumor size, inhibiting replication of cancer cells, lessening of overall tumor burden, preventing or inhibiting metastasis, ameliorating one or more symptoms associated with cancer, and prolonging survival.

The terms “detection” or “detected” as used herein includes qualitative and/or quantitative detection (measuring levels) with or without reference to a control.

The term “diagnosing”, as used herein, means the determination of the nature of a medical condition intended to identify a pathology which affects the subject from a number of collected data.

The terms “subject,” “individual,” and “patient” are used interchangeably herein to refer to a mammal, including, but not limited to, human, bovine, horse, feline, canine, rodent, or primate. In some embodiments, the individual is a human.

It is understood that embodiments of the application described herein include “consisting” and or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

It will be understood by one of ordinary skill in the art that uracil and thymine can both be represented by ‘t’, instead of ‘u’ for uracil and ‘t’ for thymine; in the context of a ribonucleic acid, it will be understood that ‘t’ is used to represent uracil unless otherwise indicated.

### Antibody Binding Affinity

Binding affinity or binding specificity of an antibody or antibody construct as described herein can be determined experimentally by methods known in the art. Such methods comprise, but are not limited to Western blots, ELISA, RIA, ECL, IRMA, EIA, BIACORE tests and peptide scans.

In some embodiments, the K_{D} of the binding between the antibody (or antibody construct) and the target antigen (e.g., CEACAM5) is about 10⁻⁷ M to about 10⁻¹² M, about 10⁻⁷ M to about 10⁻⁸ M, about 10⁻⁸ M to about 10⁻⁹ M, about 10⁻⁹ M to about 10⁻¹⁰ M, about 10⁻¹⁰ M to about 10⁻¹¹ M, about 10⁻¹¹ M to about 10⁻¹² M, about 10⁻⁷ M to about 10⁻¹² M, about 10⁻⁸ M to about 10⁻¹² M, about 10⁻⁹ M to about 10⁻¹² M, about 10⁻¹⁰ M to about 10⁻¹² M, about 10⁻⁷ M to about 10⁻¹¹ M, about 10⁸ M to about 10⁻¹¹ M, about 10⁻⁹ M to about 10⁻¹¹ M, about 10⁻⁷ M to about 10⁻¹⁰ M, about 10⁻⁸ M to about 10⁻¹⁰ M, or about 10⁻⁷ M to about 10⁻⁹ M. In some embodiments, the K_{D} of the binding between the antibody (or antibody construct) and the target antigen (e.g., CEACAM5) is less than about any one of 10⁻⁷ M, 10⁻⁸ M, 10⁻⁹ M, 10⁻¹⁰ M, 10⁻¹¹ M, or 10⁻¹² M.

In some embodiments, the Kₒₙ of the binding between the antibody (or antibody construct) and the target antigen (e.g., CEACAM5) is about 10³ M⁻¹s⁻¹ to about 10⁸ M⁻¹s⁻¹, about 10³ M⁻¹s⁻¹ to about 10⁴ M⁻¹s⁻¹, about 10⁴ M⁻¹s⁻¹ to about 10⁵ M⁻¹s⁻¹, about 10⁵ M⁻¹s⁻¹ to about 10⁶ M⁻¹s⁻¹, about 10⁶ M⁻¹s⁻¹ to about 10⁷ M⁻¹s⁻¹, or about 10⁷ M⁻¹s⁻¹ to about 10⁸ M⁻¹s⁻¹. In some embodiments, the Kₒₙ of the binding between the antibody (or antibody construct) and the target antigen (e.g., CEACAM5) is about 10³ M⁻¹s⁻¹ to about 10⁵ M⁻¹s⁻¹, about 10⁴ M⁻¹s⁻¹ to about 10⁶ M⁻¹s⁻¹, about 10⁵ M⁻¹s⁻¹ to about 10⁷ M⁻¹s⁻¹, about 10⁶ M⁻¹s⁻¹ to about 10⁸ M⁻¹s⁻¹, about 10⁴ M⁻¹s⁻¹ to about 10⁷ M⁻¹s⁻¹, or about 10⁵ M⁻¹s⁻¹ to about 10⁸ M⁻¹s⁻¹. In some embodiments, the Kₒₙ of the binding between the antibody (or antibody construct) and the target antigen (e.g., CEACAM5) is no more than about any one of 10³ M⁻¹s⁻¹, 10⁴ M⁻¹s⁻¹, 10⁵ M⁻¹s⁻¹, 10⁶ M⁻¹s⁻¹, 10⁷ M⁻¹s⁻¹ or 10⁸ M⁻¹s⁻¹.

In some embodiments, the K_{off} of the binding between the antibody (or antibody construct) and the target antigen (e.g., CEACAM5) is about 1 s⁻¹ to about 10⁻⁶ s⁻¹, about 1 s⁻¹ to about 10⁻² s⁻¹, about 10⁻² s⁻¹ to about 10⁻³ s⁻¹, about 10⁻³ s⁻¹ to about 10⁻⁴ s⁻¹, about 10⁻⁴ s⁻¹ to about 10⁻⁵ s⁻¹, about 10⁻⁵ s⁻¹ to about 10⁻⁶ s⁻¹, about 1 s⁻¹ to about 10⁻⁵ s⁻¹, about 10⁻² s⁻¹ to about 10⁻⁶ s⁻¹, about 10⁻³ s⁻¹ to about 10⁻⁶ s⁻¹, about 10⁴ s⁻¹ to about 10⁶ s⁻¹, about 10⁻² s⁻¹ to about 10⁻⁵ s⁻¹, or about 10⁻³ s⁻¹ to about 10⁻⁵ s⁻¹. In some embodiments, the K_{off} of the binding between the antibody (or antibody construct) and the target antigen (e.g., CEACAM5) is at least about any one of 1 s⁻¹, 10⁻² s⁻¹, 10⁻³ s⁻¹, 10⁻⁴ s⁻¹, 10⁻⁵ s⁻¹ or 10⁻⁶ s⁻¹.

### Chimeric or Humanized Antibodies

In some embodiments, one or more of the antibodies of the present application is a chimeric antibody. Certain chimeric antibodies are described, e.g., in U.S. Pat. No. 4,816,567; and Morrison et al., Proc. Natl. Acad. Sci. USA, 81:6851-6855 (1984)). In some embodiments, a chimeric antibody comprises a non-human variable region (e.g., a variable region derived from mouse) and a human constant region. In some embodiments, a chimeric antibody is a “class switched” antibody in which the class or subclass has been changed from that of the parent antibody. Chimeric antibodies include antigen binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); Framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Substitution, Insertion, and Deletion Variants

In some embodiments, antibody variants comprising one or more amino acid substitutions are included in the antibodies or antibody constructs described herein. Sites of interest for substitutional mutagenesis include the HVRs (or CDRs) and FRs. Conservative substitutions are shown in Table 2 under the heading of “Preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2
Amino acid substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

### Anti-CEACAM5 Single Domain Antibodies

CEACAM family members are highly glycosylated proteins with a typical N-terminal variable Ig-like domain followed by zero to six constant Ig-like domains, as well as a hydrophobic transmembrane domain with a cytoplasmic tail (CEACAM1 to CEACAM4) or a glycosylphosphatidylinositol lipid moiety (CEACAM5 to CEACAM8).

CEACAM5 belongs to the CEACAM family and is involved in intercellular contact via both homophilic and heterophilic binding (with CEACAM1 or CEACAM6). In addition to its functions in cell adhesion and migration, CEACAM5 also inhibits anoikis. As resistance to anoikis is a characteristic of cancer cells, the inhibitory effect of CEACAM5 on anoikis suggests its role in facilitating tumorigenesis and metastasis.

The members of the CEACAM family have been reported to participate in cancerous growth and invasion by acting as either tumor suppressors or poor prognostic markers for the progression of malignancies. CEACAM5 is upregulated in approximately 90% of gastrointestinal, colorectal, and pancreatic cancers and 50% of breast cancers. CEACAM5 has been applied in the clinical detection of liver metastasis, colorectal cancer, and colon cancer relapse.

The CEACAM5 (CEA) comprises one N domain followed by six C2-like domains (A1, B1, A2, B2, A3 and B3). In some embodiments, the CEACAM5 protein is a wild-type CEACAM5 (e.g., wild-type human CEACAM5). In some embodiments, the CEACAM5 protein is a natural variant CEACAM5. In some embodiments, the CEACAM5 protein is a mutant CEACAM5 (e.g., mutant human CEACAM5). The CEACAM5 protein described herein can be from various sources.

In some embodiments, the CEACAM5 protein as used herein is a human CEACAM5, such as one comprising the amino acid sequence as set forth in SEQ ID NO: 98. The A3 and B3 domains are membrane proximal domains of human CEACAM5, which consist of the amino acids at positions 499-685 of SEQ ID NO: 98, as shown in SEQ ID NO: 99. In some embodiments, the CEACAM5 protein is a non-human CEACAM5, such as derived from any of llama, horse, donkey, dog, cat, cow, sheep, pig, fish, amphibian, reptile, goat, bird, monkey, mouse, rabbit, rat, hamster, etc. In some embodiments, the CEACAM5 protein is derived from a mammal.

In one aspect, provided herein is an antibody (e.g., sdAb) specifically binding to CEACAM5 comprising CDR1, CDR2 and CDR3 of a single domain antibody of anyone selected from the group consisting of SEQ ID NOs: 1-10. In some embodiments, the CDR1, CDR2 and CDR3 are according to IMGT numbering. In some embodiments, the CDR1, CDR2 and CDR3 are according to Kabat numbering. In some embodiments, the CDR1, CDR2 and CDR3 are according to Chothia numbering. In some embodiments, the CDR1, CDR2 and CDR3 are according to Abm numbering. In some embodiments, the CDR1, CDR2 and CDR3 are according to Contact numbering.

In another aspect, provided herein is an antibody (e.g., sdAb) specifically binding to CEACAM5 comprising CDR1, CDR2 and CDR3 of a single domain antibody of anyone selected from the group consisting of SEQ ID NOs: 1-10, wherein CDR1, CDR2 and CDR3 are according to IMGT numbering. In some embodiments, the anti-CEACAM5 antibody comprises: (1) a CDR1 comprising an amino acid sequence selected from the group consisting of SEQ ID NOs: 11-20, or an amino acid sequence with one or more (e.g., 1, 2, 3, or more) amino acid alterations (e.g., substitutions, deletions, or insertions) as compared to any of SEQ ID NOs: 11-20; (2) a CDR2 comprising an amino acid sequence selected from the group consisting of SEQ ID NOs: 21-30, or an amino acid sequence with one or more (e.g., 1, 2, 3, or more) amino acid alterations (e.g., substitutions, deletions, or insertions) as compared to any of SEQ ID NOs: 21-30; and (3) a CDR3 comprising an amino acid sequence selected from the group consisting of SEQ ID NOs: 31-40, or an amino acid sequence with one or more (e.g., 1, 2, 3, or more) amino acid alterations (e.g., substitutions, deletions, or insertions) as compared to any of SEQ ID NOs: 31-40.

In some embodiments, provided herein is an antibody (e.g., sdAb) specifically binding to CEACAM5 comprising CDR1, CDR2 and CDR3 of a single domain antibody of SEQ ID NO: 1. In some embodiments, the anti-CEACAM5 antibody (e.g., sdAb) comprises CDR1, CDR2 and CDR3 of a single domain antibody of SEQ ID NO: 1, and the CDR1, CDR2 and CDR3 are according to IMGT numbering. In some embodiments, the antibody comprises: (1) a CDR1 comprising an amino acid sequence of SEQ ID NO: 11 or an amino acid sequence with one or more (e.g., 1, 2, 3, or more) amino acid alterations (e.g., substitutions, deletions, or insertions) as compared to SEQ ID NO: 11, (2) a CDR2 comprising an amino acid sequence of SEQ ID NO: 21 or an amino acid sequence with one or more (e.g., 1, 2, 3, or more) amino acid alterations (e.g., substitutions, deletions, or insertions) as compared to SEQ ID NO: 21, and (3) a CDR3 comprising an amino acid sequence of SEQ ID NO: 31 or an amino acid sequence with one or more (e.g., 1, 2, 3, or more) amino acid alterations (e.g., substitutions, deletions, or insertions) as compared to SEQ ID NO: 31. In some embodiments, the antibody comprises: (1) a CDR1 comprising an amino acid sequence of SEQ ID NO: 11, (2) a CDR2 comprising an amino acid sequence of SEQ ID NO: 21, and (3) a CDR3 comprising an amino acid sequence of SEQ ID NO: 31. In some embodiments, the anti-CEACAM5 antibody (e.g., sdAb) comprises CDR1, CDR2 and CDR3 of a single domain antibody of SEQ ID NO: 1, and the CDR1, CDR2 and CDR3 are according to Kabat numbering. In some embodiments, the antibody comprises: (1) a CDR1 comprising an amino acid sequence of SEQ ID NO: 41 or an amino acid sequence with one or more (e.g., 1, 2, 3, or more) amino acid alterations (e.g., substitutions, deletions, or insertions) as compared to SEQ ID NO: 41, (2) a CDR2 comprising an amino acid sequence of SEQ ID NO: 51 or an amino acid sequence with one or more (e.g., 1, 2, 3, or more) amino acid alterations (e.g., substitutions, deletions, or insertions) as compared to SEQ ID NO: 51, and (3) a CDR3 comprising an amino acid sequence of SEQ ID NO: 61 or an amino acid sequence with one or more (e.g., 1, 2, 3, or more) amino acid alterations (e.g., substitutions, deletions, or insertions) as compared to SEQ ID NO: 61. In some embodiments, the antibody comprises: (1) a CDR1 comprising an amino acid sequence of SEQ ID NO: 41, (2) a CDR2 comprising an amino acid sequence of SEQ ID NO: 51, and (3) a CDR3 comprising an amino acid sequence of SEQ ID NO: 61. In some embodiments, the antibody comprises an amino acid sequence having at least about 70% (e.g., at least about any of 80%, 85%, 90%, 95%, 96%, 97%, 98%, 99%, or more) identity with SEQ ID NO: 1. In some embodiments, the anti-CEACAM5 antibody comprises an amino acid sequence of SEQ ID NO: 1.

In some embodiments, provided herein is an antibody (e.g., sdAb) specifically binding to CEACAM5 comprising CDR1, CDR2 and CDR3 of a single domain antibody of SEQ ID NO: 2. In some embodiments, the anti-CEACAM5 antibody (e.g., sdAb) comprises CDR1, CDR2 and CDR3 of a single domain antibody of SEQ ID NO: 2, and CDR1, CDR2 and CDR3 are according to IMGT numbering. In some embodiments, the antibody comprises: (1) a CDR1 comprising an amino acid sequence of SEQ ID NO: 12 or an amino acid sequence with one or more (e.g., 1, 2, 3, or more) amino acid alterations (e.g., substitutions, deletions, or insertions) as compared to SEQ ID NO: 12, (2) a CDR2 comprising an amino acid sequence of SEQ ID NO: 22 or an amino acid sequence with one or more (e.g., 1, 2, 3, or more) amino acid alterations (e.g., substitutions, deletions, or insertions) as compared to SEQ ID NO: 22, and (3) a CDR3 comprising an amino acid sequence of SEQ ID NO: 32 or an amino acid sequence with one or more (e.g., 1, 2, 3, or more) amino acid alterations (e.g., substitutions, deletions, or insertions) as compared to SEQ ID NO: 32. In some embodiments, the antibody comprises: (1) a CDR1 comprising an amino acid sequence of SEQ ID NO: 12, (2) a CDR2 comprising an amino acid sequence of SEQ ID NO: 22, and (3) a CDR3 comprising an amino acid sequence of SEQ ID NO: 32. In some embodiments, the anti-CEACAM5 antibody (e.g., sdAb) comprises CDR1, CDR2 and CDR3 of a single domain antibody of SEQ ID NO: 2, and the CDR1, CDR2 and CDR3 are according to Kabat numbering. In some embodiments, the antibody comprises: (1) a CDR1 comprising an amino acid sequence of SEQ ID NO: 42 or an amino acid sequence with one or more (e.g., 1, 2, 3, or more) amino acid alterations (e.g., substitutions, deletions, or insertions) as compared to SEQ ID NO: 42, (2) a CDR2 comprising an amino acid sequence of SEQ ID NO: 52 or an amino acid sequence with one or more (e.g., 1, 2, 3, or more) amino acid alterations (e.g., substitutions, deletions, or insertions) as compared to SEQ ID NO: 52, and (3) a CDR3 comprising an amino acid sequence of SEQ ID NO: 62 or an amino acid sequence with one or more (e.g., 1, 2, 3, or more) amino acid alterations (e.g., substitutions, deletions, or insertions) as compared to SEQ ID NO: 62. In some embodiments, the antibody comprises: (1) a CDR1 comprising an amino acid sequence of SEQ ID NO: 42, (2) a CDR2 comprising an amino acid sequence of SEQ ID NO: 52, and (3) a CDR3 comprising an amino acid sequence of SEQ ID NO: 62. In some embodiments, the antibody comprises an amino acid sequence having at least about 70% (e.g., at least about any of 80%, 85%, 90%, 95%, 96%, 97%, 98%, 99%, or more) identity with SEQ ID NO: 2. In some embodiments, the anti-CEACAM5 antibody comprises an amino acid sequence of SEQ ID NO: 2.

In some embodiments, provided herein is an antibody (e.g., sdAb) specifically binding to CEACAM5 comprising CDR1, CDR2 and CDR3 of a single domain antibody of SEQ ID NO: 3. In some embodiments, the anti-CEACAM5 antibody (e.g., sdAb) comprises CDR1, CDR2 and CDR3 of a single domain antibody of SEQ ID NO: 3, and CDR1, CDR2 and CDR3 are according to IMGT numbering. In some embodiments, the antibody comprises: (1) a CDR1 comprising an amino acid sequence of SEQ ID NO: 13 or an amino acid sequence with one or more (e.g., 1, 2, 3, or more) amino acid alterations (e.g., substitutions, deletions, or insertions) as compared to SEQ ID NO: 13, (2) a CDR2 comprising an amino acid sequence of SEQ ID NO: 23 or an amino acid sequence with one or more (e.g., 1, 2, 3, or more) amino acid alterations (e.g., substitutions, deletions, or insertions) as compared to SEQ ID NO: 23, and (3) a CDR3 comprising an amino acid sequence of SEQ ID NO: 33 or an amino acid sequence with one or more (e.g., 1, 2, 3, or more) amino acid alterations (e.g., substitutions, deletions, or insertions) as compared to SEQ ID NO: 33. In some embodiments, the antibody comprises: (1) a CDR1 comprising an amino acid sequence of SEQ ID NO: 13. (2) a CDR2 comprising an amino acid sequence of SEQ ID NO: 23, and (3) a CDR3 comprising an amino acid sequence of SEQ ID NO: 33. In some embodiments, the anti-CEACAM5 antibody (e.g., sdAb) comprises CDR1, CDR2 and CDR3 of a single domain antibody of SEQ ID NO: 3, and the CDR1, CDR2 and CDR3 are according to Kabat numbering. In some embodiments, the antibody comprises: (1) a CDR1 comprising an amino acid sequence of SEQ ID NO: 43 or an amino acid sequence with one or more (e.g., 1, 2, 3, or more) amino acid alterations (e.g., substitutions, deletions, or insertions) as compared to SEQ ID NO: 43, (2) a CDR2 comprising an amino acid sequence of SEQ ID NO: 53 or an amino acid sequence with one or more (e.g., 1, 2, 3, or more) amino acid alterations (e.g., substitutions, deletions, or insertions) as compared to SEQ ID NO: 53, and (3) a CDR3 comprising an amino acid sequence of SEQ ID NO: 63 or an amino acid sequence with one or more (e.g., 1, 2, 3, or more) amino acid alterations (e.g., substitutions, deletions, or insertions) as compared to SEQ ID NO: 63. In some embodiments, the antibody comprises: (1) a CDR1 comprising an amino acid sequence of SEQ ID NO: 43, (2) a CDR2 comprising an amino acid sequence of SEQ ID NO: 53, and (3) a CDR3 comprising an amino acid sequence of SEQ ID NO: 63. In some embodiments, the antibody comprises an amino acid sequence having at least about 70% (e.g., at least about any of 80%, 85%, 90%, 95%, 96%, 97%, 98%, 99%, or more) identity with SEQ ID NO: 3. In some embodiments, the anti-CEACAM5 antibody comprises an amino acid sequence of SEQ ID NO: 3.

In some embodiments, provided herein is an antibody (e.g., sdAb) specifically binding to CEACAM5 comprising CDR1, CDR2 and CDR3 of a single domain antibody of SEQ ID NO: 4. In some embodiments, the anti-CEACAM5 antibody (e.g., sdAb) comprises CDR1, CDR2 and CDR3 of a single domain antibody of SEQ ID NO: 4, and CDR1, CDR2 and CDR3 are according to IMGT numbering. In some embodiments, the antibody comprises: (1) a CDR1 comprising an amino acid sequence of SEQ ID NO: 14 or an amino acid sequence with one or more (e.g., 1, 2, 3, or more) amino acid alterations (e.g., substitutions, deletions, or insertions) as compared to SEQ ID NO: 14, (2) a CDR2 comprising an amino acid sequence of SEQ ID NO: 24 or an amino acid sequence with one or more (e.g., 1, 2, 3, or more) amino acid alterations (e.g., substitutions, deletions, or insertions) as compared to SEQ ID NO: 24, and (3) a CDR3 comprising an amino acid sequence of SEQ ID NO: 34 or an amino acid sequence with one or more (e.g., 1, 2, 3, or more) amino acid alterations (e.g., substitutions, deletions, or insertions) as compared to SEQ ID NO: 34. In some embodiments, the antibody comprises: (1) a CDR1 comprising an amino acid sequence of SEQ ID NO: 14, (2) a CDR2 comprising an amino acid sequence of SEQ ID NO: 24, and (3) a CDR3 comprising an amino acid sequence of SEQ ID NO: 34. In some embodiments, the anti-CEACAM5 antibody (e.g., sdAb) comprises CDR1, CDR2 and CDR3 of a single domain antibody of SEQ ID NO: 4, and the CDR1, CDR2 and CDR3 are according to Kabat numbering. In some embodiments, the antibody comprises: (1) a CDR1 comprising an amino acid sequence of SEQ ID NO: 44 or an amino acid sequence with one or more (e.g., 1, 2, 3, or more) amino acid alterations (e.g., substitutions, deletions, or insertions) as compared to SEQ ID NO: 44, (2) a CDR2 comprising an amino acid sequence of SEQ ID NO: 54 or an amino acid sequence with one or more (e.g., 1, 2, 3, or more) amino acid alterations (e.g., substitutions, deletions, or insertions) as compared to SEQ ID NO: 54, and (3) a CDR3 comprising an amino acid sequence of SEQ ID NO: 64 or an amino acid sequence with one or more (e.g., 1, 2, 3, or more) amino acid alterations (e.g., substitutions, deletions, or insertions) as compared to SEQ ID NO:64. In some embodiments, the antibody comprises: (1) a CDR1 comprising an amino acid sequence of SEQ ID NO: 44, (2) a CDR2 comprising an amino acid sequence of SEQ ID NO: 54, and (3) a CDR3 comprising an amino acid sequence of SEQ ID NO: 64. In some embodiments, the antibody comprises an amino acid sequence having at least about 70% (e.g., at least about any of 80%, 85%, 90%, 95%, 96%, 97%, 98%, 99%, or more) identity with SEQ ID NO: 4. In some embodiments, the anti-CEACAM5 antibody comprises an amino acid sequence of SEQ ID NO: 4.

In some embodiments, provided herein is an antibody (e.g., sdAb) specifically binding to CEACAM5 comprising CDR1, CDR2 and CDR3 of a single domain antibody of SEQ ID NO: 5. In some embodiments, the anti-CEACAM5 antibody (e.g., sdAb) comprises CDR1, CDR2 and CDR3 of a single domain antibody of SEQ ID NO: 5, and CDR1, CDR2 and CDR3 are according to IMGT numbering. In some embodiments, the antibody comprises: (1) a CDR1 comprising an amino acid sequence of SEQ ID NO: 15 or an amino acid sequence with one or more (e.g., 1, 2, 3, or more) amino acid alterations (e.g., substitutions, deletions, or insertions) as compared to SEQ ID NO: 15, (2) a CDR2 comprising an amino acid sequence of SEQ ID NO: 25 or an amino acid sequence with one or more (e.g., 1, 2, 3, or more) amino acid alterations (e.g., substitutions, deletions, or insertions) as compared to SEQ ID NO: 25, and (3) a CDR3 comprising an amino acid sequence of SEQ ID NO: 35 or an amino acid sequence with one or more (e.g., 1, 2, 3, or more) amino acid alterations (e.g., substitutions, deletions, or insertions) as compared to SEQ ID NO: 35. In some embodiments, the antibody comprises: (1) a CDR1 comprising an amino acid sequence of SEQ ID NO: 15, (2) a CDR2 comprising an amino acid sequence of SEQ ID NO: 25, and (3) a CDR3 comprising an amino acid sequence of SEQ ID NO: 35. In some embodiments, the anti-CEACAM5 antibody (e.g., sdAb) comprises CDR1, CDR2 and CDR3 of a single domain antibody of SEQ ID NO: 5, and the CDR1, CDR2 and CDR3 are according to Kabat numbering. In some embodiments, the antibody comprises: (1) a CDR1 comprising an amino acid sequence of SEQ ID NO: 45 or an amino acid sequence with one or more (e.g., 1, 2, 3, or more) amino acid alterations (e.g., substitutions, deletions, or insertions) as compared to SEQ ID NO: 45, (2) a CDR2 comprising an amino acid sequence of SEQ ID NO: 55 or an amino acid sequence with one or more (e.g., 1, 2, 3, or more) amino acid alterations (e.g., substitutions, deletions, or insertions) as compared to SEQ ID NO: 55, and (3) a CDR3 comprising an amino acid sequence of SEQ ID NO: 65 or an amino acid sequence with one or more (e.g., 1, 2, 3, or more) amino acid alterations (e.g., substitutions, deletions, or insertions) as compared to SEQ ID NO: 65. In some embodiments, the antibody comprises: (1) a CDR1 comprising an amino acid sequence of SEQ ID NO: 45, (2) a CDR2 comprising an amino acid sequence of SEQ ID NO:55, and (3) a CDR3 comprising an amino acid sequence of SEQ ID NO: 65. In some embodiments, the antibody comprises an amino acid sequence having at least about 70% (e.g., at least about any of 80%, 85%, 90%, 95%, 96%, 97%, 98%, 99%, or more) identity with SEQ ID NO: 5. In some embodiments, the anti-CEACAM5 antibody comprises an amino acid sequence of SEQ ID NO: 5.

In some embodiments, provided herein is an antibody (e.g., sdAb) specifically binding to CEACAM5 comprising CDR1, CDR2 and CDR3 of a single domain antibody of SEQ ID NO: 6. In some embodiments, the anti-CEACAM5 antibody (e.g., sdAb) comprises CDR1, CDR2 and CDR3 of a single domain antibody of SEQ ID NO: 6, and CDR1, CDR2 and CDR3 are according to IMGT numbering. In some embodiments, the antibody comprises: (1) a CDR1 comprising an amino acid sequence of SEQ ID NO: 16 or an amino acid sequence with one or more (e.g., 1, 2, 3, or more) amino acid alterations (e.g., substitutions, deletions, or insertions) as compared to SEQ ID NO: 16, (2) a CDR2 comprising an amino acid sequence of SEQ ID NO: 26 or an amino acid sequence with one or more (e.g., 1, 2, 3, or more) amino acid alterations (e.g., substitutions, deletions, or insertions) as compared to SEQ ID NO: 26, and (3) a CDR3 comprising an amino acid sequence of SEQ ID NO: 36 or an amino acid sequence with one or more (e.g., 1, 2, 3, or more) amino acid alterations (e.g., substitutions, deletions, or insertions) as compared to SEQ ID NO: 36. In some embodiments, the antibody comprises: (1) a CDR1 comprising an amino acid sequence of SEQ ID NO: 16. (2) a CDR2 comprising an amino acid sequence of SEQ ID NO: 26, and (3) a CDR3 comprising an amino acid sequence of SEQ ID NO: 36. In some embodiments, the anti-CEACAM5 antibody (e.g., sdAb) comprises CDR1, CDR2 and CDR3 of a single domain antibody of SEQ ID NO: 6, and the CDR1, CDR2 and CDR3 are according to Kabat numbering. In some embodiments, the antibody comprises: (1) a CDR1 comprising an amino acid sequence of SEQ ID NO: 46 or an amino acid sequence with one or more (e.g., 1, 2, 3, or more) amino acid alterations (e.g., substitutions, deletions, or insertions) as compared to SEQ ID NO: 46, (2) a CDR2 comprising an amino acid sequence of SEQ ID NO: 56 or an amino acid sequence with one or more (e.g., 1, 2, 3, or more) amino acid alterations (e.g., substitutions, deletions, or insertions) as compared to SEQ ID NO: 56, and (3) a CDR3 comprising an amino acid sequence of SEQ ID NO: 66 or an amino acid sequence with one or more (e.g., 1, 2, 3, or more) amino acid alterations (e.g., substitutions, deletions, or insertions) as compared to SEQ ID NO: 66. In some embodiments, the antibody comprises: (1) a CDR1 comprising an amino acid sequence of SEQ ID NO: 46, (2) a CDR2 comprising an amino acid sequence of SEQ ID NO: 56, and (3) a CDR3 comprising an amino acid sequence of SEQ ID NO: 66. In some embodiments, the antibody comprises an amino acid sequence having at least about 70% (e.g., at least about any of 80%, 85%, 90%, 95%, 96%, 97%, 98%, 99%, or more) identity with SEQ ID NO: 6. In some embodiments, the anti-CEACAM5 antibody comprises an amino acid sequence of SEQ ID NO: 6.

In some embodiments, provided herein is an antibody (e.g., sdAb) specifically binding to CEACAM5 comprising CDR1, CDR2 and CDR3 of a single domain antibody of SEQ ID NO: 7. In some embodiments, the anti-CEACAM5 antibody (e.g., sdAb) comprises CDR1, CDR2 and CDR3 of a single domain antibody of SEQ ID NO: 7, and CDR1, CDR2 and CDR3 are according to IMGT numbering. In some embodiments, the antibody comprises: (1) a CDR1 comprising an amino acid sequence of SEQ ID NO: 17 or an amino acid sequence with one or more (e.g., 1, 2, 3, or more) amino acid alterations (e.g., substitutions, deletions, or insertions) as compared to SEQ ID NO: 17, (2) a CDR2 comprising an amino acid sequence of SEQ ID NO: 27 or an amino acid sequence with one or more (e.g., 1, 2, 3, or more) amino acid alterations (e.g., substitutions, deletions, or insertions) as compared to SEQ ID NO: 27, and (3) a CDR3 comprising an amino acid sequence of SEQ ID NO: 37 or an amino acid sequence with one or more (e.g., 1, 2, 3, or more) amino acid alterations (e.g., substitutions, deletions, or insertions) as compared to SEQ ID NO: 37. In some embodiments, the antibody comprises: (1) a CDR1 comprising an amino acid sequence of SEQ ID NO: 17, (2) a CDR2 comprising an amino acid sequence of SEQ ID NO: 27, and (3) a CDR3 comprising an amino acid sequence of SEQ ID NO: 37. In some embodiments, the anti-CEACAM5 antibody (e.g., sdAb) comprises CDR1, CDR2 and CDR3 of a single domain antibody of SEQ ID NO: 7, and the CDR1, CDR2 and CDR3 are according to Kabat numbering. In some embodiments, the antibody comprises: (1) a CDR1 comprising an amino acid sequence of SEQ ID NO: 47 or an amino acid sequence with one or more (e.g., 1, 2, 3, or more) amino acid alterations (e.g., substitutions, deletions, or insertions) as compared to SEQ ID NO: 47, (2) a CDR2 comprising an amino acid sequence of SEQ ID NO: 57 or an amino acid sequence with one or more (e.g., 1, 2, 3, or more) amino acid alterations (e.g., substitutions, deletions, or insertions) as compared to SEQ ID NO: 57, and (3) a CDR3 comprising an amino acid sequence of SEQ ID NO: 67 or an amino acid sequence with one or more (e.g., 1, 2, 3, or more) amino acid alterations (e.g., substitutions, deletions, or insertions) as compared to SEQ ID NO: 67. In some embodiments, the antibody comprises: (1) a CDR1 comprising an amino acid sequence of SEQ ID NO: 47, (2) a CDR2 comprising an amino acid sequence of SEQ ID NO: 57, and (3) a CDR3 comprising an amino acid sequence of SEQ ID NO: 67. In some embodiments, the antibody comprises an amino acid sequence having at least about 70% (e.g., at least about any of 80%, 85%, 90%, 95%, 96%, 97%, 98%, 99%, or more) identity with SEQ ID NO: 7. In some embodiments, the anti-CEACAM5 antibody comprises an amino acid sequence of SEQ ID NO: 7.

In some embodiments, provided herein is an antibody (e.g., sdAb) specifically binding to CEACAM5 comprising CDR1, CDR2 and CDR3 of a single domain antibody of SEQ ID NO: 8. In some embodiments, the anti-CEACAM5 antibody (e.g., sdAb) comprises CDR1, CDR2 and CDR3 of a single domain antibody of SEQ ID NO: 8, and CDR1, CDR2 and CDR3 are according to IMGT numbering. In some embodiments, the antibody comprises: (1) a CDR1 comprising an amino acid sequence of SEQ ID NO: 18 or an amino acid sequence with one or more (e.g., 1, 2, 3, or more) amino acid alterations (e.g., substitutions, deletions, or insertions) as compared to SEQ ID NO: 18, (2) a CDR2 comprising an amino acid sequence of SEQ ID NO: 28 or an amino acid sequence with one or more (e.g., 1, 2, 3, or more) amino acid alterations (e.g., substitutions, deletions, or insertions) as compared to SEQ ID NO: 28, and (3) a CDR3 comprising an amino acid sequence of SEQ ID NO: 38 or an amino acid sequence with one or more (e.g., 1, 2, 3, or more) amino acid alterations (e.g., substitutions, deletions, or insertions) as compared to SEQ ID NO: 38. In some embodiments, the antibody comprises: (1) a CDR1 comprising an amino acid sequence of SEQ ID NO: 18, (2) a CDR2 comprising an amino acid sequence of SEQ ID NO: 28, and (3) a CDR3 comprising an amino acid sequence of SEQ ID NO: 38. In some embodiments, the anti-CEACAM5 antibody (e.g., sdAb) comprises CDR1, CDR2 and CDR3 of a single domain antibody of SEQ ID NO: 8, and the CDR1, CDR2 and CDR3 are according to Kabat numbering. In some embodiments, the antibody comprises: (1) a CDR1 comprising an amino acid sequence of SEQ ID NO: 48 or an amino acid sequence with one or more (e.g., 1, 2, 3, or more) amino acid alterations (e.g., substitutions, deletions, or insertions) as compared to SEQ ID NO: 48, (2) a CDR2 comprising an amino acid sequence of SEQ ID NO: 58 or an amino acid sequence with one or more (e.g., 1, 2, 3, or more) amino acid alterations (e.g., substitutions, deletions, or insertions) as compared to SEQ ID NO: 58, and (3) a CDR3 comprising an amino acid sequence of SEQ ID NO: 68 or an amino acid sequence with one or more (e.g., 1, 2, 3, or more) amino acid alterations (e.g., substitutions, deletions, or insertions) as compared to SEQ ID NO: 68. In some embodiments, the antibody comprises: (1) a CDR1 comprising an amino acid sequence of SEQ ID NO: 48, (2) a CDR2 comprising an amino acid sequence of SEQ ID NO: 58, and (3) a CDR3 comprising an amino acid sequence of SEQ ID NO: 68. In some embodiments, the antibody comprises an amino acid sequence having at least about 70% (e.g., at least about any of 80%, 85%, 90%, 95%, 96%, 97%, 98%, 99%, or more) identity with SEQ ID NO: 8. In some embodiments, the anti-CEACAM5 antibody comprises an amino acid sequence of SEQ ID NO: 8.

In some embodiments, provided herein is an antibody (e.g., sdAb) specifically binding to CEACAM5 comprising CDR1, CDR2 and CDR3 of a single domain antibody of SEQ ID NO: 9. In some embodiments, the anti-CEACAM5 antibody (e.g., sdAb) comprises CDR1, CDR2 and CDR3 of a single domain antibody of SEQ ID NO: 9, and CDR1, CDR2 and CDR3 are according to IMGT numbering. In some embodiments, the antibody comprises: (1) a CDR1 comprising an amino acid sequence of SEQ ID NO: 19 or an amino acid sequence with one or more (e.g., 1, 2, 3, or more) amino acid alterations (e.g., substitutions, deletions, or insertions) as compared to SEQ ID NO: 19, (2) a CDR2 comprising an amino acid sequence of SEQ ID NO: 29 or an amino acid sequence with one or more (e.g., 1, 2, 3, or more) amino acid alterations (e.g., substitutions, deletions, or insertions) as compared to SEQ ID NO: 29, and (3) a CDR3 comprising an amino acid sequence of SEQ ID NO: 39 or an amino acid sequence with one or more (e.g., 1, 2, 3, or more) amino acid alterations (e.g., substitutions, deletions, or insertions) as compared to SEQ ID NO: 39. In some embodiments, the antibody comprises: (1) a CDR1 comprising an amino acid sequence of SEQ ID NO: 19, (2) a CDR2 comprising an amino acid sequence of SEQ ID NO: 29, and (3) a CDR3 comprising an amino acid sequence of SEQ ID NO: 39. In some embodiments, the anti-CEACAM5 antibody (e.g., sdAb) comprises CDR1, CDR2 and CDR3 of a single domain antibody of SEQ ID NO: 9, and the CDR1, CDR2 and CDR3 are according to Kabat numbering. In some embodiments, the antibody comprises: (1) a CDR1 comprising an amino acid sequence of SEQ ID NO: 49 or an amino acid sequence with one or more (e.g., 1, 2, 3, or more) amino acid alterations (e.g., substitutions, deletions, or insertions) as compared to SEQ ID NO: 49. (2) a CDR2 comprising an amino acid sequence of SEQ ID NO: 59 or an amino acid sequence with one or more (e.g., 1, 2, 3, or more) amino acid alterations (e.g., substitutions, deletions, or insertions) as compared to SEQ ID NO: 59, and (3) a CDR3 comprising an amino acid sequence of SEQ ID NO: 69 or an amino acid sequence with one or more (e.g., 1, 2, 3, or more) amino acid alterations (e.g., substitutions, deletions, or insertions) as compared to SEQ ID NO: 69. In some embodiments, the antibody comprises: (1) a CDR1 comprising an amino acid sequence of SEQ ID NO: 49, (2) a CDR2 comprising an amino acid sequence of SEQ ID NO: 59, and (3) a CDR3 comprising an amino acid sequence of SEQ ID NO: 69. In some embodiments, the antibody comprises an amino acid sequence having at least about 70% (e.g., at least about any of 80%, 85%, 90%, 95%, 96%, 97%, 98%, 99%, or more) identity with SEQ ID NO: 9. In some embodiments, the anti-CEACAM5 antibody comprises an amino acid sequence of SEQ ID NO: 9.

In some embodiments, provided herein is an antibody (e.g., sdAb) specifically binding to CEACAM5 comprising CDR1, CDR2 and CDR3 of a single domain antibody of SEQ ID NO: 10. In some embodiments, the anti-CEACAM5 antibody (e.g., sdAb) comprises CDR1, CDR2 and CDR3 of a single domain antibody of SEQ ID NO: 10, and CDR1, CDR2 and CDR3 are according to IMGT numbering. In some embodiments, the antibody comprises: (1) a CDR1 comprising an amino acid sequence of SEQ ID NO: 20 or an amino acid sequence with one or more (e.g., 1, 2, 3, or more) amino acid alterations (e.g., substitutions, deletions, or insertions) as compared to SEQ ID NO: 20, (2) a CDR2 comprising an amino acid sequence of SEQ ID NO: 30 or an amino acid sequence with one or more (e.g., 1, 2, 3, or more) amino acid alterations (e.g., substitutions, deletions, or insertions) as compared to SEQ ID NO: 30, and (3) a CDR3 comprising an amino acid sequence of SEQ ID NO: 40 or an amino acid sequence with one or more (e.g., 1, 2, 3, or more) amino acid alterations (e.g., substitutions, deletions, or insertions) as compared to SEQ ID NO: 40. In some embodiments, the antibody comprises: (1) a CDR1 comprising an amino acid sequence of SEQ ID NO: 20. (2) a CDR2 comprising an amino acid sequence of SEQ ID NO: 30, and (3) a CDR3 comprising an amino acid sequence of SEQ ID NO: 40. In some embodiments, the anti-CEACAM5 antibody (e.g., sdAb) comprises CDR1, CDR2 and CDR3 of a single domain antibody of SEQ ID NO: 10, and the CDR1, CDR2 and CDR3 are according to Kabat numbering. In some embodiments, the antibody comprises: (1) a CDR1 comprising an amino acid sequence of SEQ ID NO: 50 or an amino acid sequence with one or more (e.g., 1, 2, 3, or more) amino acid alterations (e.g., substitutions, deletions, or insertions) as compared to SEQ ID NO: 50, (2) a CDR2 comprising an amino acid sequence of SEQ ID NO: 60 or an amino acid sequence with one or more (e.g., 1, 2, 3, or more) amino acid alterations (e.g., substitutions, deletions, or insertions) as compared to SEQ ID NO: 60, and (3) a CDR3 comprising an amino acid sequence of SEQ ID NO: 70 or an amino acid sequence with one or more (e.g., 1, 2, 3, or more) amino acid alterations (e.g., substitutions, deletions, or insertions) as compared to SEQ ID NO: 70. In some embodiments, the antibody comprises: (1) a CDR1 comprising an amino acid sequence of SEQ ID NO: 50, (2) a CDR2 comprising an amino acid sequence of SEQ ID NO: 60, and (3) a CDR3 comprising an amino acid sequence of SEQ ID NO: 70. In some embodiments, the antibody comprises an amino acid sequence having at least about 70% (e.g., at least about any of 80%, 85%, 90%, 95%, 96%, 97%, 98%, 99%, or more) identity with SEQ ID NO: 10. In some embodiments, the anti-CEACAM5 antibody comprises an amino acid sequence of SEQ ID NO: 10.

In some embodiments, the anti-CEACAM5 antibody (e.g., sdAb) is a humanized antibody. In some embodiments, the humanized anti-CEACAM5 antibody is derived from anyone selected from the group consisting of SEQ ID NOs: 1-10.

In some embodiments, the humanized anti-CEACAM5 antibody (e.g., sdAb) is derived from SEQ ID NO: 1 and comprises an amino acid sequence having at least about 70% (e.g., at least about any of 80%, 85%, 90%, 95%, 96%, 97%, 98%, 99%, or more) identity with anyone selected from the group consisting of SEQ ID NOs: 71-73. In some embodiments, the humanized anti-CEACAM5 antibody (e.g., sdAb) comprises an amino acid sequence of anyone selected from the group consisting of SEQ ID NOs: 71-73.

In some embodiments, the humanized anti-CEACAM5 antibody (e.g., sdAb) is derived from SEQ ID NO: 2 and comprises an amino acid sequence having at least about 70% (e.g., at least about any of 80%, 85%, 90%, 95%, 96%, 97%, 98%, 99%, or more) identity with SEQ ID NO: 74 or 75. In some embodiments, the humanized anti-CEACAM5 antibody (e.g., sdAb) comprises an amino acid sequence of SEQ ID NO: 74 or 75.

In some embodiments, the humanized anti-CEACAM5 antibody (e.g., sdAb) is derived from SEQ ID NO: 3 and comprises an amino acid sequence having at least about 70% (e.g., at least about any of 80%, 85%, 90%, 95%, 96%, 97%, 98%, 99%, or more) identity with SEQ ID NO: 76 or 77. In some embodiments, the humanized anti-CEACAM5 antibody (e.g., sdAb) comprises an amino acid sequence of SEQ ID NO: 76 or 77.

In some embodiments, the humanized anti-CEACAM5 antibody (e.g., sdAb) is derived from SEQ ID NO: 4 and comprises an amino acid sequence having at least about 70% (e.g., at least about any of 80%, 85%, 90%, 95%, 96%, 97%, 98%, 99%, or more) identity with anyone selected from the group consisting of SEQ ID NOs: 78-80. In some embodiments, the humanized anti-CEACAM5 antibody (e.g., sdAb) comprises an amino acid sequence of anyone selected from the group consisting of SEQ ID NOs: 78-80.

In some embodiments, the humanized anti-CEACAM5 antibody (e.g., sdAb) is derived from SEQ ID NO: 5 and comprises an amino acid sequence having at least about 70% (e.g., at least about any of 80%, 85%, 90%, 95%, 96%, 97%, 98%, 99%, or more) identity with SEQ ID NO: 81 or 82. In some embodiments, the humanized anti-CEACAM5 antibody (e.g., sdAb) comprises an amino acid sequence of SEQ ID NO: 81 or 82.

In some embodiments, the humanized anti-CEACAM5 antibody (e.g., sdAb) is derived from SEQ ID NO: 6 and comprises an amino acid sequence having at least about 70% (e.g., at least about any of 80%, 85%, 90%, 95%, 96%, 97%, 98%, 99%, or more) identity with SEQ ID NO: 83 or 84. In some embodiments, the humanized anti-CEACAM5 antibody (e.g., sdAb) comprises an amino acid sequence of SEQ ID NO: 83 or 84.

In some embodiments, the humanized anti-CEACAM5 antibody (e.g., sdAb) is derived from SEQ ID NO: 7 and comprises an amino acid sequence having at least about 70% (e.g., at least about any of 80%, 85%, 90%, 95%, 96%, 97%, 98%, 99%, or more) identity with SEQ ID NO: 85 or 86. In some embodiments, the humanized anti-CEACAM5 antibody (e.g., sdAb) comprises an amino acid sequence of SEQ ID NO: 85 or 86.

In some embodiments, the humanized anti-CEACAM5 antibody (e.g., sdAb) is derived from SEQ ID NO: 8 and comprises an amino acid sequence having at least about 70% (e.g., at least about any of 80%, 85%, 90%, 95%, 96%, 97%, 98%, 99%, or more) identity with SEQ ID NO: 87 or 88. In some embodiments, the humanized anti-CEACAM5 antibody (e.g., sdAb) comprises an amino acid sequence of SEQ ID NO: 87 or 88.

In some embodiments, the humanized anti-CEACAM5 antibody (e.g., sdAb) is derived from SEQ ID NO: 9 and comprises an amino acid sequence having at least about 70% (e.g., at least about any of 80%, 85%, 90%, 95%, 96%, 97%, 98%, 99%, or more) identity with anyone selected from the group consisting of SEQ ID NOs: 89-94. In some embodiments, the humanized anti-CEACAM5 antibody (e.g., sdAb) comprises an amino acid sequence of anyone selected from the group consisting of SEQ ID NOs: 89-94.

In some embodiments, the humanized anti-CEACAM5 antibody (e.g., sdAb) is derived from SEQ ID NO: 10 and comprises an amino acid sequence having at least about 70% (e.g., at least about any of 80%, 85%, 90%, 95%, 96%, 97%, 98%, 99%, or more) identity with anyone selected from the group consisting of SEQ ID NOs: 95-97. In some embodiments, the humanized anti-CEACAM5 antibody (e.g., sdAb) comprises an amino acid sequence of anyone selected from the group consisting of SEQ ID NOs: 95-97.

In some embodiments, the anti-CEACAM5 antibody described herein is a single domain antibody (e.g., VHH).

### Antibody Constructs

In another aspect, provided herein is an antibody construct comprising any of the anti-CEACAM5 antibodies (e.g., sdAbs, such as any of SEQ ID NOs: 1-10 and 71-97) described herein. In some embodiments, the antibody construct further comprises one or more (e.g., 1, 2, 3 or more) additional antibody moieties. In some embodiments, the antibody construct is a multispecific (e.g., bispecific) antibody construct capable of specifically binding to CEACAM5 and one or more (e.g., one) additional targets. In some embodiments, the one or more additional targets are different from CEACAM5. In some embodiments, the one or more additional targets are different CEACAM5 epitopes from the one bound by the anti-CEACAM5 antibodies (e.g., sdAbs) described herein. In some embodiments, one or more of the additional targets are the same CEACAM5 epitope as the one bound by the anti-CEACAM5 antibodies (e.g., sdAbs) described herein. In some embodiments, the antibody construct is a bispecific antibody construct capable of specifically binding to CEACAM5 and a second target. In some embodiments, the second target is different from CEACAM5. In some embodiments, the second target is also CEACAM5, e.g., a different CEACAM5 epitope. In some embodiments, the antibody construct is a multivalent (e.g., bivalent) but monospecific antibody construct, e.g., the one or more additional antibody moieties all bind to the same CEACAM5 epitope bound by the anti-CEACAM5 antibodies (e.g., sdAbs) described herein. In some embodiments, the antibody construct comprises two or more anti-CEACAM5 antibodies (e.g., sdAbs) described herein arranged in tandem, which can be monospecific or multispecific.

The term “multispecific” or “bispecific” as used herein refers to an antibody with one part of the antibody binds to one epitope on an antigen whereas the additional part (or the second part) binds to a different epitope on the antigen, or on a different antigen. The different epitope is typically present on a different antigen. In some embodiments, the bispecific antibody is an antibody that comprises parts of two different antibodies and consequently binds to two different types of antigens. One arm of the bispecific antibody typically contains a variable domain of one antibody and the other arm contains a variable domain of another antibody. In some embodiments, one arm of the bispecific antibody typically contains a variable domain (e.g., sdAb or VHH) targeting CEACAM5 and the other arm contains a variable domain of another antibody targeting the second target.

In some embodiments, the additional target is a tumor antigen. In some embodiments, the additional target is an immune cell (e.g., T cell, NK cell, or B cell) specific antigen. In some embodiments, the additional target is an immune checkpoint molecule. In some embodiments, the one or more additional antibody moieties within the antibody construct are selected from the group consisting of a full-length antibody, a Fab, a Fab′, a (Fab′) 2, an Fv, a single chain Fv (scFv), an scFv-scFv, a minibody, a diabody, or an sdAb.

In some embodiments, the antibody construct comprises an Fc fragment. In some embodiments, the Fc fragment is derived from any of IgG1, IgG2, IgG3 and IgG4. In some embodiments, the Fc fragment further comprises one or more mutations to alter the function of the Fc fragment, for example, to reduce or enhance ADCC (Antibody-dependent cell-mediated cytotoxicity), CDC (Complement dependent cytotoxicity) and/or ADCP (Antibody-dependent cellular phagocytosis) function of the Fc fragment. In some embodiments, the antibody construct is a heavy chain only antibody (HCAb) comprising any of the anti-CEACAM5 antibodies (e.g., sdAbs) described herein fused to an Fc fragment.

### Conjugates

In another aspect, provided herein is a conjugate comprising: (1) any of the antibodies specifically binding to CEACAM5 (e.g., sdAbs, such as any of SEQ ID NOs: 1-10 and 71-97) or the antibody constructs described herein, and (2) a drug. In some embodiments, the drug is selected from the group consisting of chemotherapeutic agent, toxin, hormone, enzyme, immunomodulator, chelator, imaging agent, nanoparticle, detection label (e.g., fluorescent label), and radioisotope.

### Nucleic Acids, Vectors, Host Cell, and Methods of Producing CEACAM5 Antibodies

Nucleic acid molecules encoding any of the antibodies specifically binding to CEACAM5 (e.g., anti-CEACAM5 sdAbs, such as any of SEQ ID NOs: 1-10 and 71-97) or the antibody constructs described herein are also contemplated. In some embodiments, provided is a nucleic acid encoding any of the anti-CEACAM5 antibodies (e.g., sdAbs) or antibody constructs described herein. In some embodiments, the nucleic acid comprises a polynucleotide sequence having at least about 70% (e.g., at least about any of 80%, 85%, 90%, 95%, 96%, 97%, 98%, 99%, or more) identity with anyone selected from the group consisting of SEQ ID NOs: 100-109. In some embodiments, the nucleic acid comprises a polynucleotide sequence of anyone selected from the group consisting of SEQ ID NOs: 100-109.

Also provided are vectors comprising any of the nucleic acids described herein. In some embodiments, the expression of the anti-CEACAM5 antibodies (e.g., sdAbs) or antibody constructs described herein can be achieved by inserting the nucleic acid described herein into an appropriate expression vector, such that the nucleic acid is operably linked to 5′ and 3′ regulatory elements, including for example a promoter (e.g., a constitutive, regulatable, tissue-specific promoter) and a 3′ untranslated region (UTR). The vectors can be suitable for replication and integration in eukaryotic host cells. The vectors can also be suitable for expression in prokaryotic cells (e.g., E. coli). Typical cloning and expression vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 base pairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

In some embodiments, the expression of the nucleic acid(s) encoding the antibody specifically binding to CEACAM5 or antibody construct described herein is inducible. In some embodiments, the nucleic acid(s) is operably linked to an inducible promoter, including any inducible promoter known in the art. In some embodiments, the nucleic acid(s) has been engineered to encode an epitope tag, e.g., to facilitate purification or detection of the polypeptide. Exemplary epitope tags include, but are not limited to, e.g., 6×His (also known as His-tag or hexahistidine tag), FLAG, HA, Myc, V5, GFP (green fluorescent protein, e.g., enhanced green fluorescent protein or EGFP), GST (glutathione-S-transferase), β-GAL (β-galactosidase), Luciferase, MBP (Maltose Binding Protein), RFP (Red Fluorescence Protein), and VSV-G (Vesicular Stomatitis Virus Glycoprotein).

The anti-CEACAM5 antibodies (e.g., sdAbs) or antibody constructs described herein can be produced by any means known in the art. Exemplary techniques for polypeptide production are described below; however, these exemplary techniques are provided for illustrative purposes only and are not intended to be limiting. Also see Example 1 for production method.

The anti-CEACAM5 antibodies (e.g., sdAbs) or antibody constructs can be produced using recombinant methods. For recombinant production, nucleic acid encoding the anti-CEACAM5 antibody (e.g., sdAb) or antibody construct is isolated and inserted into a replicable vector for further cloning (amplification of the DNA) or for expression. DNA encoding the antibody or antibody construct may be readily isolated and sequenced using conventional procedures (e.g., by using oligonucleotide probes that are capable of binding specifically to genes encoding the antibody or antibody construct). The vector components generally include, but are not limited to, one or more of the following: a signal sequence, an origin of replication, one or more marker genes, an enhancer element, a promoter, and a transcription termination sequence.

In some embodiments, there is provided a method of making any of the anti-CEACAM5 antibodies (e.g., sdAbs, such as any of SEQ ID NOs: 1-10 and 71-97) or antibody constructs described herein, comprising: (a) culturing a host cell comprising any of the isolated nucleic acids or vectors described herein, or any of the host cells described herein, under a condition effective to express the encoded antibody or antibody construct; and (b) obtaining the expressed antibody or antibody construct from the host cell.

### Chimeric Antigen Receptors and Engineered T-Cell Receptors

“Chimeric antigen receptor” or “CAR” as used herein refers to genetically engineered receptors, which can be used to graft one or more antigen specificity onto immune effector cells, such as T cells or NK cells. Some CARs are also known as “artificial T-cell receptors,” “chimeric T cell receptors,” or “chimeric immune receptors.” In some embodiments, the CAR comprises an extracellular antigen binding domain specific for one or more antigens (such as CEACAM5), a transmembrane domain, and an intracellular signaling domain (e.g., of a T cell, such as CD39), and or other receptors (e.g., CD28 or 4-1BB co-stimulatory signaling domain). “CAR-T” refers to a T cell that expresses a CAR. “CAR-NK” refers to an NK cell that expresses a CAR.

In another aspect, provided herein is a CAR comprising any of the anti-CEACAM5 antibody (e.g., sdAb, such as any of SEQ ID NOs: 1-10 and 71-97) or antibody construct described herein. In another aspect, provided herein is an anti-CEACAM5 CAR comprising: (i) an extracellular antigen binding domain comprising one or more of the anti-CEACAM5 antibody (e.g., sdAb, such as any of SEQ ID NOs: 1-10 and 71-97) or antibody construct described herein; (ii) a transmembrane domain, and (iii) an intracellular signaling domain.

As used herein, the term “T-cell receptor” or “TCR” refers to a molecule on the surface of a T cell or T lymphocyte that is responsible for recognizing an antigen. TCR is a heterodimer which is composed of two different protein chains. In some embodiments, the TCR consists of an alpha (α) chain and a beta (β) chain and is referred as αβ TCR. αβ TCR recognizes antigenic peptides degraded from protein bound to major histocompatibility complex molecules (MHC) at the cell surface. In some embodiments, the TCR consists of a gamma (γ) and a delta (δ) chain and is referred as γδ TCR.

In another aspect, provided herein is an engineered TCR comprising any of the anti-CEACAM5 antibody (e.g., sdAb, such as any of SEQ ID NOs: 1-10 and 71-97) or antibody construct described herein. In some embodiments, the engineered TCR comprises: (i) an extracellular antigen binding domain comprising one or more anti-CEACAM5 antibody (e.g., sdAb, such as any of SEQ ID NOs: 1-10 and 71-97) or antibody construct described herein, and (ii) a transmembrane domain comprising a first transmembrane module and a second transmembrane module; wherein the extracellular antigen binding domain is at the N-terminus of the transmembrane domain (e.g., fused to one or both transmembrane modules). In some embodiments, the transmembrane domain is derived from TCRαβ. In some embodiments, the transmembrane domain is derived from TCRδγ. In some embodiments, the engineered TCR further comprises a stabilization domain at the N-terminus of the transmembrane domain (e.g., between the extracellular antigen binding domain and the transmembrane domain). In some embodiments, the stabilization domain comprises Cα-Cβ. In some embodiments, the stabilization domain comprises Cδ-Cγ. In some embodiments, the engineered TCR further comprises an intracellular domain derived from TCRαβ or TCRδγ at the C-terminus of the transmembrane domain. In some embodiments, the engineered TCR is capable of recruiting at least one TCR-associated signaling molecule selected from the group consisting of CD3δε, CD3γε, and ζζ.

In some embodiments, also provided are cells (e.g., immune effector cells) expressing the CAR or engineered TCR described herein. In some embodiments, the cell is an immune cell, such as immune effector cell. In some embodiments, the cell is selected from the group consisting of a T cell (e.g., cytotoxic T cell), an NK cell, a peripheral blood mononuclear cell (PBMC), a hematopoietic stem cell, a pluripotent stem cell, or an embryonic stem cell.

### Pharmaceutical Composition

In another aspect, provided herein is a pharmaceutical composition comprising any of the anti-CEACAM5 antibodies (e.g., sdAbs, such as any of SEQ ID NOs: 1-10 and 71-97), antibody constructs, isolated nucleic acids, vectors, conjugates, or cells expressing CAR or engineered TCR as described herein, and a pharmaceutically acceptable excipient.

Any suitable excipient can be used in the pharmaceutical composition of the present application. In some embodiments, the excipient includes but is not limited to diluents; carriers; excipients; stabilizers; buffers such as phosphate, citrate, and other organic acids buffers; antioxidants including ascorbic acid and d methionine; preservatives as such octadecyldimethylbenzyl ammonium chloride, hexamethonium chloride, enzalkonium chloride, benzethonium chloride, phenol, butyl or benzyl alcohol, alkyl parabens such as methyl or propyl paraben, catechol, resorcinol, cyclohexanol, 3-pentanol, and m-cresol); low molecular weight (less than about 10 residues) polypeptides; proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

### Methods of Treatment and Diagnosis

Also provided are methods of treating a disorder (e.g., CEACAM5-associated disorder) in a subject (e.g., human) in need thereof, comprising administrating an effective amount of any of the anti-CEACAM5 antibodies (e.g., sdAbs, such as any of SEQ ID NOs: 1-10 and 71-97), antibody constructs, conjugates, vectors (e.g., viral vector), cells expressing CAR or engineered TCR described herein, or pharmaceutical compositions thereof to the subject. In some embodiments, the method comprises administrating any of the pharmaceutical compositions described herein to the subject. In some embodiments, there is provided a use of an effective amount of any of the anti-CEACAM5 antibodies (e.g., sdAbs, such as any of SEQ ID NOs: 1-10 and 71-97), antibody constructs, conjugates, vectors (e.g., viral vector), cells expressing CAR or engineered TCR described herein, or pharmaceutical compositions thereof, in the manufacture of a medicament for treating a disorder (e.g., CEACAM5-associated disorder) in a subject (e.g., human) in need thereof. In some embodiments, there is provided a use of an effective amount of any of the pharmaceutical compositions described herein in the manufacture of a medicament for treating a disorder (e.g., CEACAM5-associated disorder) in a subject (e.g., human) in need thereof. In some embodiments, the disorder is associated with undesired presence of CEACAM5+ cells.

In some embodiments, the disorder (e.g., CEACAM5-associated disorder) is a proliferative disorder. In some embodiments, the disorder is a cancer, such as CEACAM5+ cancer. Numerous studies have shown that CEACAM5, identical to the originally identified CEA, is highly expressed on the surface of colorectal, gastric, lung, breast, prostate, ovary, cervix, and bladder tumor cells and weakly expressed in few normal epithelial tissues such as columnar epithelial and goblet cells in colon, mucous neck cells in the stomach and squamous epithelial cells in esophagus and cervix (Hammarstrom et al, 2002, in “Tumor markers, Physiology, Pathobiology, Technology and Clinical Applications” Eds. Diamandis E. P. et al., AACC Press, Washington pp 375). In some embodiments, the CEACAM5+ cancer is selected from the group consisting of colorectal cancer, stomach cancer, lung cancer, uterus cervix cancer, pancreas cancer, oesophagus cancer, ovary cancer, thyroid cancer, bladder cancer, endometrium cancer, breast cancer, liver cancer (e.g., cholangiocarcinoma), prostate cancer, and skin cancer. In some embodiments, the CEACAM5+ cancer is gastric cancer.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. The examples below are intended to be purely exemplary of the application and should therefore not be considered to limit the application in any way. The following examples and detailed description are offered by way of illustration and not by way of limitation.

### Example 1: Generation of Single Domain Antibodies (sdAb) Specifically Binding to CEACAM5

### 1.1 Animal Immunization and Phage Library Construction

Two alpacas were subcutaneously injected with human CEACAM5 (SEQ ID NO: 98) and human A3B3 domain (SEQ ID NO: 99) every 2 weeks for 6 doses, 100 μg to 200 μg antigen per dose. Blood samples were collected at different time points, and induction of an antigen specific humoral immune response was verified by ELISA and FCM using the serum prepared from the blood samples.

7 days after the final immunization, 50 mL blood sample was collected from each animal and approximately 1×10⁸ peripheral blood mononuclear cells (PBMCs) were obtained from the blood sample by density gradient centrifugation on Ficoll-Paque PLUS (GE Healthcare, Little Chalfont, UK). Total RNA was extracted from the PBMCs and transcribed into cDNA using an oligo-dT primer and SuperScript III First-Strand Synthesis SuperMix System (Invitrogen, Carlsbad, CA, USA) according to the manufacturers' recommendations.

The purified cDNA was then used as template to amplify the repertoire of Ig heavy chain-encoding gene segments by using signal peptide domain specific primers and CH2 domain specific primers. Fragments of approximately 700 bp (representing heavy-chain IgGs that lack a CH1 domain) were isolated from agarose gel and purified by QIAquick Gel Extraction Kit (Qiagen, Hilden, Germany). The purified fragments were further used as templates to amplify the VHH repertoire by using framework1 (FR1) and framework4 (FR4) specific primer pairs.

After amplification, purified VIIII genes were cloned into phagemid vector pFL249, and electrotransformed into E. coli TG1. After transformation, the TG1 cells were cultured in SOC medium at 37° C. for 1 hour, then inoculated onto plates containing solid 2YT medium supplemented with 100 μg/mL Carb and 1% (w/v) glucose and cultured at 37° C. overnight. The next day, the colonies were scraped into liquid 2YT medium supplemented with 1/3 (v/v) of 80% glycerol and stored at −80° C. The library size was about 1×10⁸, VHH insert ratio was above 95%, and phage display ratio is above 85%.

### 1.2 Antibody Panning and Screening

The constructed library was subject to three rounds of panning, followed by three rounds of screening of binding capability via ELISA and FCM using sdAb-expressing phage supernatant. 45 screened clones were expressed in E. coli and were further screened by ELISA and FCM to have 10 clones finally selected. The sequences of the 10 clones are as shown in Table 3.

TABLE 3
Sequences of exemplary anti-CEACAM5 single domain antibodies

| SEQ | |
|---|---|
| ID | |
| NO: | Sequence |
| 1 | EVQLVESGGGLVQAGGSLRLSCVASTRIAGMGWYRAPPGKGRELVASISEEGTTTYAVPVEGRFT |
| | VSRDNVKNMLHLQMNSLKPEDTAMYYCVARRTFPSVVEGVDYWGQGTQVTVSS |
| 2 | QLQLVESGGGLVQPGGSLRLSCAVSGDRWDYYAIGWIRQAPGKEREGVSCISSSGRTTNYADSVK |
| | GRFTISRDNAKNTVYLQMNSLKPEDTAVYYCSTEFTGWDRCGFDEYDHRGQGTQVTVSS |
| 3 | EVDLVESGGGLVQPGGSLRLSCAASGFTFSSYYMSWVRQPPGKGIEWVSEISSGGGSTNYAVPVEG |
| | RFTISRDNAKNTLSLQMNSLKPEDTALYYCARGSHADFNSWGQGTQVTVSS |
| 4 | EVQLVESGGGLVQPGESLRLSCAASGFSFSSYAMSWVRQPPGMGIEWVAEIDSGGGSTDYAVPVK |
| | GRFTISRDNVKNTLYLQMNSLKPEDTAVFYCAKGTHADFGSWGQGTQVTVSS |
| 5 | EVDLVESGGGLVQAGGSLRLSCAPSGRTSSLDTMAWFRQAPGKEREFVASTNWNRGGASYADSV |
| | KGRFTISRDATKNTVYLQINSLEPEDTAVYYCAASTRGIVSDNYWGQGTQVTVSS |
| 6 | EVQLVESGGGLVQPGGSLRLSCAASRGTFSRLDMGWFRQAPGKEREFVASSTWSGGTAYATDSV |
| | KTRFTISRDSAKTTVYLQMNSLRPEDTAVYYCAAGLSLEQHTPPYWGQGTQVTVSS |
| 7 | EVQLVESGGGLVQAGGSLRLSCAASGRTFSRDVMGWFRQAPGKEREFVAVINWTDSTYYLDSVK |
| | GRFTISRENAENTVYLQMNSLKPEDTAIYYCATGIGGLYRRPSAYKYWGQGTQVTVSS |
| 8 | AVQLVESGGGLVQAGGSLRLSCAASGRTFSSYWLGWFRQVPGEEREFVAAISWSGSGVVTNYADS |
| | VKGRFTISRDDGNNALYLQMSNLNPEDTAVYYCAARLGTYYKDYARSGAYDTWGQGTQVTVSS |
| 9 | QVQLVESGGGLVQPGESLRLSCAASGFTFSSYAMSWVRQPPGKGIEWVAEIDSGGDRTDYAVPVK |
| | GRFTISRDNVENTLYLQMNSLKPEDTAVFYCAKGTHADFGSWGQGTLVTVSS |
| 10 | EVDLMESGGGLVQPGGSLRLSCTASGDTLDYYAIDWFRQAPGKEREGVSCISSRGGTTNYADAVK |
| | GRFTISRDNAKNTVYLQMNSLRPEDTAVYYCAAEFTGWAVCNFSRKSGEYDYWGQGTQVTVSS |

### Example 2. Characterization of Exemplary Anti-CEACAM5 Single Domain Antibodies

2.1 Binding Affinity of Anti-CEACAM5 sdAbs Via Surface Plasmon Resonance (SPR)

Exemplary anti-CEACAM5 sdAbs were diluted with 1×HBS-EP buffer. Human CEACAM5 A3B3 domain was captured on anti-human Fc capturing Ab-immobilized CM5 chip. Anti-CEACAM5 sdAb samples were then injected with a flow rate of 10 μl/min for 180 sec, followed by dissociation for 800 sec. The binding curves were locally fitted with Biacore®T200 analysis software (BIAevaluation) using a 1:1 Langmuir binding model. The result is shown in Table 4. Based on the SPR results, it can be seen that all the tested clones are potent CEACAM5 A3B3 binders with K_{D} ranging between about 10⁻¹⁰ to about 10⁻⁹ M.

TABLE 4
Binding affinities of exemplary anti-CEACAM5 sdAbs

| SEQ ID NO: | Kₐ (1/Ms) | K_{d} (1/s) | K_{D} (M) |
|---|---|---|---|
| 1 | 1.231E+05 | 4.460E−05 | 3.620E−10 |
| 2 | 1.871E+06 | 9.972E−04 | 5.330E−10 |
| 3 | 4.136E+05 | 3.201E−03 | 7.741E−09 |
| 4 | 1.553E+05 | 3.106E−04 | 2.000E−09 |
| 5 | 2.139E+05 | 2.083E−03 | 9.738E−09 |
| 6 | 1.624E+05 | 1.262E−03 | 7.772E−09 |
| 7 | 6.875E+05 | 1.304E−02 | 1.897E−08 |
| 8 | 1.274E+05 | 2.430E−03 | 1.906E−08 |
| 9 | 1.231E+05 | 4.460E−05 | 7.320E−10 |
| 10 | 2.799E+05 | 1.368E−02 | 4.888E−08 |

2.2 Binding Affinities of Exemplary Anti-CEACAM5 sdAbs with MKN45 Cells

MKN45 cells (human gastric cancer cell line) expressing CEACAM5 were used to evaluate the binding affinity of the exemplary anti-CEACAM5 sdAbs. Briefly, 50 μL MKN45 cells at density of 3×10⁶ cells/mL were incubated with 50 μL anti-CEACAM5 sdAbs at 4° C. for 2 hours. Incubation with human IgG1 or no antibody served as negative control. After incubation, cells were washed three times with 400 μL FACS buffer, resuspended with PE-anti human Fc 2nd antibody (Biolegend 410708, 1:100 dilution) and incubated at 4° C. for 60 min. Then cells were collected and subject to FACS analysis. The result is shown in Table 5 below. Based on the FACS results, the tested clones showed significant binding capabilities to CEACAM5-expressing MKN45 cells.

TABLE 5
Geometric mean MFI of exemplary
anti-CEACAM5 sdAbs (300 nm)

| ID | MFI |
|---|---|
| Human IgG1 isotype | 12.9 |
| 2^{nd} antibody only | 11.1 |
| Blank | 10.6 |
| SEQ ID NO: 1 | 292 |
| SEQ ID NO: 2 | 565 |
| SEQ ID NO: 4 | 394 |
| SEQ ID NO: 5 | 292 |
| SEQ ID NO: 6 | 647 |
| SEQ ID NO: 8 | 636 |
| SEQ ID NO: 9 | 382 |

### Example 3. Internalization of Exemplary Anti-CEACAM5 Single Domain Antibodies

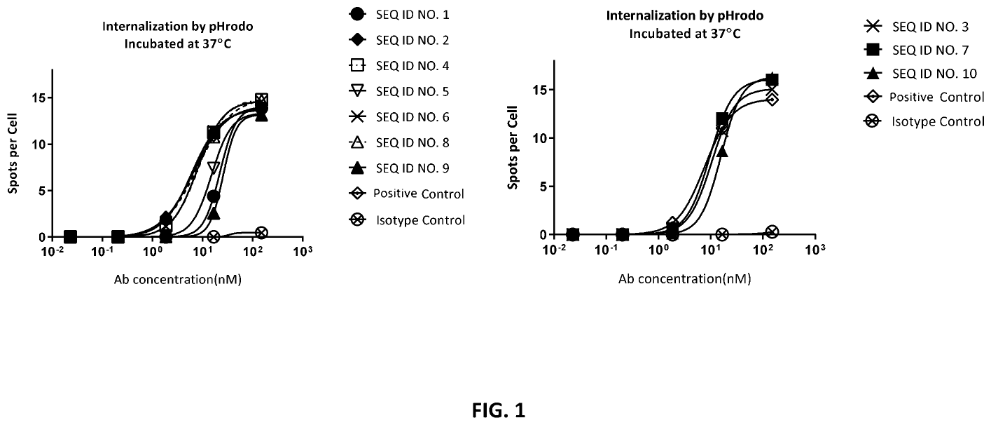

Anti-cMyc antibody was labeled with pHrodo iFL Red STP ester amine reactive dye and purified by Zeba™ spin desalting column according to the manufacturer's instructions. MKN45 cells expressing CEACAM5 were seeded on 96-well plates at a number of 1×10⁴ cells/well in complete culture medium containing 10% FBS, and cultured at 37° C., 5% CO₂ overnight. Exemplary anti-CEACAM5 sdAbs at different concentrations were mixed with the pHrodo iFL Red/anti-cMyc antibody conjugates at a ratio of 1:1.2 (unit: mol/L). CEACAM5+MKN45 cells were then incubated with the mixture at 37° C. for 24 hours. Incubation with tusamitamab (anti-CEACAM5, Sanofi) served as positive control. Incubation with human IgG1 isotype antibody served as negative control. After incubation, the cells were treated with 100 μL Hoechst 33342 and Calcein AM diluted in DPBS at 37° C. for 15 min. The fluorescence intensity of MKN45 cells was analyzed by Perkin Elmer Operetta CLS High-Content Analysis System. The internalization result is shown in FIG. 1, indicating that the tested clones were all internalized into MKN45 cells after 24 hours.

### Example 4. Antibody Binning

Exemplary anti-CEACAM5 sdAbs were diluted with 1×HBS-EP buffer. Human CEACAM5 A3B3 domain was captured on anti-human Fc capturing Ab-immobilized CM5 chip. Then an anti-CEACAM5 sdAb sample and a competitive anti-CEACAM5 sdAb were sequentially co-injected with a flow rate of 30 μl/min for 120×2 sec, followed by dissociation for 120 sec. The raw data were manually analyzed in BIAcore® T200 BIAevaluation software and the results were shown in Table 6. Based on Table 6, it can be seen that anti-CEACAM5 sdAbs with SEQ ID NOs: 1, 2, 3, 7, 9, and 10 substantially bind to the same epitope in CEACAM5 antigen; anti-CEACAM5 sdAbs with SEQ ID NOs: 5-6 substantially bind to the same epitope in CEACAM5 antigen; and anti-CEACAM5 sdAbs with SEQ ID NOs: 4 and 8 substantially bind to the same epitope in CEACAM5 antigen.

TABLE 6
Antibody binning

| SEQ ID NO: | 3 | 7 | 10 | 9 | 2 | 1 | 6 | 5 | 8 | 4 |
|---|---|---|---|---|---|---|---|---|---|---|
| 3 | Y | | | | | Y | | N | | N |
| 7 | | Y | | | | Y | | N | | N |
| 10 | | | Y | | | Y | | N | | N |
| 9 | | | | Y | Y | Y | N | N | N | N |
| 2 | | | | Y | Y | Y | N | N | N | N |
| 1 | Y | Y | Y | Y | Y | Y | N | N | N | N |
| 6 | | | | N | N | N | Y | Y | N | N |
| 5 | N | N | N | N | N | N | Y | Y | N | N |
| 8 | | | | N | N | N | N | N | Y | Y |
| 4 | N | N | N | N | N | N | N | N | Y | Y |

Y indicates substantial binding on the same epitope of the two clones based on binning results, and
N indicates substantial binding on different epitopes of the two clones based on binning results

### Example 5. Humanization of Exemplary Anti-CEACAM5 Single Domain Antibodies

Selected anti-CEACAM5 sdAbs were further subject to humanization. Briefly, the amino acid sequences of the clones in Table 3 were mapped against available database of human Ig gene sequences to obtain the best-matching human germline Ig gene sequences. Then coding sequences of CDR1, CDR2, and CDR3 of each selected sdAbs were grafted onto the framework of its best-matching human germline Ig gene, respectively, to obtain humanized sdAbs as shown in Table 7 below.

TABLE 7
Humanized anti-CEACAM5 sdAbs

| SEQ | |
|---|---|
| ID | |
| NO: | Sequence |
| 71 | EVQLVESGGGLVQPGGSLRLSCAASTRIAGMSW99FRQAPGKGRELVASISEEGTTTYADSVKGRFTIS |
| | RDNSTNTLYLQMNSLKPEDTAVYYCVARRTFPSVVEGVDYWGQGTLVTVSS |
| 72 | QVQLVESGGGLIQPGGSLRLSCAASTRIAGMGWYRAPPGKGRELVASISEEGTTTYADSVKGRFTIS |
| | RDNAKNTLYLQMNSLRAEDTAVYYCVARRTFPSVVEGVDYWGQGTQVTVSS |
| 73 | QVQLVESGGGLVQPGGSLRLSCAASTRIAGMSWYRQAPGKGRELVASISEEGTTTYADSVEGRFTI |
| | SRDNAKNTLYLQMNSLRAEDTAVYYCVARRTFPSVVEGVDYWGQGTLVTVSS |
| 74 | QVQLVESGGGLVQPGGSLRLSCAASGDRWDYYAIGWIRQAPGKEREGVSCISSSGRTTNYADSVK |
| | GRFTISRDNAKNTLYLQMNSLRAEDTAVYYCSTEFTGWDRCGFDEYDHRGQGTQVTVSS |
| 75 | QVQLVESGGGLVQPGGSLRLSCAASGDRWDYYAMSWIRQAPGKEREGVSCISSSGRTTNYADSVK |
| | GRFTISRDNARNTLYLQMNSLRAEDTAVYYCSTEFTGWDRCGFDEYDHRGQGTQVTVSS |
| 76 | QVQLVESGGGLVQPGGSLRLSCAASGFTFSSYYMSWVRQPPGKGIEWVSEISSGGGSTNYADSVK |
| | GRFTISRDNAKNTLYLQMNSLRAEDTAVYYCARGSHADFNSWGQGTQVTVSS |
| 77 | QVQLVESGGGLVQPGGSLRLSCAASGFTFSSYYMYWVRQAPGKGLEWVSEISSGGGSTNYADSVK |
| | GRFTISRDNARNTLYLQMNSLRAEDTAVYYCARGSHADFNSWGQGTQVTVSS |
| 78 | QVQLVESGGGLVQPGGSLRLSCAASGFSFSSYAMSWVRQPPGMGIEWVAEIDSGGGSTDYADSVK |
| | GRFTISRDNAKNTLYLQMNSLRAEDTAVYYCAKGTHADFGSWGQGTQVTVSS |
| 79 | EVQLVESGGGLVQPGGSLRLSCAASGFSFSSYAMSWVRQPPGMGIEWVAEIDSGGGSTDYADSVK |
| | GRFTISRDNAKNTLYLQMNSLRAEDTAVYYCAAGTHADFGSWGQGTQVTVSS |
| 80 | EVQLVESGGGLVQPGGSLRLSCAASGFSFSSYAMSWFRQPPGMGRELVAEIDSGGGSTDYADSVK |
| | GRFTISRDNAKNTLYLQMNSLRAEDTAVYYCAKGTHADFGSWGQGTQVTVSS |
| 81 | EVQLVESGGGVVRPGGSLRLSCAASGRTSSLDTMAWFRQAPGKEREFVASTNWNRGGASYADSV |
| | KGRFTISRDNAKNSLYLQMNSLRAEDTALYYCAASTRGIVSDNYWGQGTLVTVSS |
| 82 | EVQLVESGGGVVRPGGSLRLSCAASGRTSSLDTMAWFRQAPGKEREFVASTNWNRGGASYADSV |
| | KGRFTISRDNARNSLYLQMNSLRAEDTALYYCAASTRGIVSDNYWGQGTLVTVSS |
| 83 | QVQLVESGGGLVQPGGSLRLSCAASRGTFSRLDMGWFRQAPGKEREFVASSTWSGGTAYYADSV |
| | KGRFTISRDNAKNTLYLQMNSLRAEDTAVYYCAAGLSLEQHTPPYWGQGTQVTVSS |
| 84 | QVQLVESGGGLVQPGGSLRLSCAASRGTFSRLDMGWFRQAPGKEREFVASSTWSGGTAYYADSV |
| | KGRFTISRDNARNTLYLQMNSLRAEDTAVYYCAAGLSLEQHTPPYWGQGTQVTVSS |
| 85 | EVQLVESGGGLVQPGGSLRLSCAASGRTFSRDVMGWFRQAPGKEREFVAVINWTDSTYYADSVK |
| | GRFTISRDNAKNTLYLQMNSLRAEDTAVYYCATGIGGLYRRPSAYKYWGQGTQVTVSS |
| 86 | EVQLVESGGGLVQPGGSLRLSCAASGRTFSRDVMGWFRQAPGKEREFVAVINWTDSTYYADSVK |
| | GRFTISRDNAENTLYLQMNSLRAEDTAVYYCATGIGGLYRRPSAYRYWGQGTQVTVSS |
| 87 | AVQLVESGGGLVQPGGSLRLSCAASGRTFSSYWLGWFRQVPGEEREFVAAISWSGSGVVTNYADS |
| | VKGRFTISRDNAKNTLYLQMNSLRAEDTAVYYCAARLGTYYKDYARSGAYDTWGQGTQVTVSS |
| 88 | AVQLVESGGGLVQPGGSLRLSCAASGRTFSSYWLGWFRQVPGEEREFVAAISWSGSGVVTNYADS |
| | VKGRFTISRDNAKNTLYLQMNSLRAEDTAVYYCAARLGTYYRDYARSGAYDTWGQGTQVTVSS |
| 89 | QVQLVESGGGLVQPGGSLRLSCAASGFTFSSYAMSWVRQPPGKGIEWVAEIDSGGDRTDYADSVK |
| | GRFTISRDNAKNTLYLQMNSLRAEDTAVYYCAKGTHADFGSWGQGTLVTVSS |
| 90 | EVQLVESGGGLVQPGGSLRLSCAASGFTFSSYAMSWFRQPPGKEREGVAEIDSGGDRTDYADSVK |
| | GRFTISRDNSTNTLYLQMNSLKPEDTAVYYCAAGTHADFGSWGQGTLVTVSS |
| 91 | EVQLVESGGGLVQPGGSLRLSCAASGFTFSSYAMSWFRQPPGKEREGVAEIDSGGDRTDYADSVR |
| | GRFTISRDNSTNTLYLQMNSLKPEDTAVYYCAAGTHADFGSWGQGTLVTVSS |
| 92 | QVQLVESGGGLVQPGESLRLSCAASGFTFSSYAMSWVRQPPGKGIEWVAEIDSGGDRTDYAVPVK |
| | GRFTISRDNVENTLYLQMNSLKPEDTAVFYCAAGTHADFGSWGQGTLVTVSS |
| 93 | QVQLLESGGGLVQPGGSLRLSCAASGFTFSSYAMSWVRQPPGKGIEWVAEIDSGGDRTDYADSVK |
| | GRFTISRDNSRNTLYLQMNSLRAEDTAVYYCAAGTHADFGSWGRGTLVTVSS |
| 94 | QVQLLESGGGLVQPGGSLRLSCAASGFTFSSYAMSWVRQPPGNQIEWVAEIDSGGDRTDYADSVK |
| | GRFTISRDNSKNTLYLQMNSLRAEDTAVYYCAAGTHADFGSWGRGTLVTVSS |
| 95 | QVQLVESGGGLVQPGGSLRLSCTASGDTLDYYAIDWFRQAPGKEREGVSCISSRGGTTNYADSVK |
| | GRFTISRDNAKNTLYLQMNSLRAEDTAVYYCAAEFTGWAVCNFSRKSGEYDYWGQGTQVTVSS |
| 96 | EVQLVESGGGLVQPGGSLRLSCTASGDTLDYYAMHWFRQAPGKEREGVSCISSRGGTTNYADSVK |
| | GRFTISRDNAKNTLYLQMNSLRAEDTAVYYCAAEFTGWAVCNFSRKSGEYDYWGQGTQVTVSS |
| 97 | QVQLLESGGGLVQPGGSLRLSCAASGDTLDYYAMSWFRQAPGKEREGVSCISSRGGTTNYADSVK |
| | GRFTISRDNARNTLYLQMNSLRAEDTAVYYCAAEFTGWAVCNFSRRSGEYDYWGQGTQVTVSS |

### Example 6. Characterization of Humanized Anti-CEACAM5 Single Domain Antibodies

Exemplary humanized anti-CEACAM5 sdAbs are diluted with 1×HBS-EP buffer. Human CEACAM5 A3B3 domain is captured on anti-human Fc capturing Ab-immobilized CM5 chip. Then humanized anti-CEACAM5 sdAb samples are injected with a flow rate of 10 pl/min for 180 sec, followed by dissociation for 800 sec. The binding curves are fitted with Biacore®T200 analysis software (BIAevaluation) using a 1:1 Langmuir binding model.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. For example, due to codon redundancy, changes can be made in the underlying DNA sequence without affecting the protein sequence. Moreover, due to biological functional equivalency considerations, changes can be made in protein structure without affecting the biological action in kind or amount. Thus, it is intended that the present disclosure cover the modifications and variations of this disclosure provided they come within the scope of the appended claims and their equivalents.

SEQUENCE LISTING

| SEQ | |
|---|---|
| ID | |
| NO: | Sequence |
| 1 | EVQLVESGGGLVQAGGSLRLSCVASTRIAGMGWYRAPPGKGRELVASISEEGTTTYAVPVEGRFT |
| | VSRDNVKNMLHLQMNSLKPEDTAMYYCVARRTFPSVVEGVDYWGQGTQVTVSS |
| 2 | QLQLVESGGGLVQPGGSLRLSCAVSGDRWDYYAIGWIRQAPGKEREGVSCISSSGRTTNYADSVK |
| | GRFTISRDNAKNTVYLQMNSLKPEDTAVYYCSTEFTGWDRCGFDEYDHRGQGTQVTVSS |
| 3 | EVDLVESGGGLVQPGGSLRLSCAASGFTFSSYYMSWVRQPPGKGIEWVSEISSGGGSTNYAVPVEG |
| | RFTISRDNAKNTLSLQMNSLKPEDTALYYCARGSHADFNSWGQGTQVTVSS |
| 4 | EVQLVESGGGLVQPGESLRLSCAASGFSFSSYAMSWVRQPPGMGIEWVAEIDSGGGSTDYAVPVK |
| | GRFTISRDNVKNTLYLQMNSLKPEDTAVFYCAKGTHADFGSWGQGTQVTVSS |
| 5 | EVDLVESGGGLVQAGGSLRLSCAPSGRTSSLDTMAWFRQAPGKEREFVASTNWNRGGASYADSV |
| | KGRFTISRDATKNTVYLQINSLEPEDTAVYYCAASTRGIVSDNYWGQGTQVTVSS |
| 6 | EVQLVESGGGLVQPGGSLRLSCAASRGTFSRLDMGWFRQAPGKEREFVASSTWSGGTAYATDSVK |
| | TRFTISRDSAKTTVYLQMNSLRPEDTAVYYCAAGLSLEQHTPPYWGQGTQVTVSS |
| 7 | EVQLVESGGGLVQAGGSLRLSCAASGRTFSRDVMGWFRQAPGKEREFVAVINWTDSTYYLDSVK |
| | GRFTISRENAENTVYLQMNSLKPEDTAIYYCATGIGGLYRRPSAYKYWGQGTQVTVSS |
| 8 | AVQLVESGGGLVQAGGSLRLSCAASGRTFSSYWLGWFRQVPGEEREFVAAISWSGSGVVTNYADS |
| | VKGRFTISRDDGNNALYLQMSNLNPEDTAVYYCAARLGTYYKDYARSGAYDTWGQGTQVTVSS |
| 9 | QVQLVESGGGLVQPGESLRLSCAASGFTFSSYAMSWVRQPPGKGIEWVAEIDSGGDRTDYAVPVK |
| | GRFTISRDNVENTLYLQMNSLKPEDTAVFYCAKGTHADFGSWGQGTLVTVSS |
| 10 | EVDLMESGGGLVQPGGSLRLSCTASGDTLDYYAIDWFRQAPGKEREGVSCISSRGGTTNYADAVK |
| | GRFTISRDNAKNTVYLQMNSLRPEDTAVYYCAAEFTGWAVCNFSRKSGEYDYWGQGTQVTVSS |
| 11 | TRIAG |
| 12 | GDRWDYYA |
| 13 | GFTFSSYY |
| 14 | GFSFSSYA |
| 15 | GRTSSLDT |
| 16 | RGTFSRLD |
| 17 | GRTFSRDV |
| 18 | GRTFSSYW |
| 19 | GFTFSSYA |
| 20 | GDTLDYYA |
| 21 | ISEEGTT |
| 22 | ISSSGRTT |
| 23 | ISSGGGST |
| 24 | IDSGGGST |
| 25 | TNWNRGGA |
| 26 | STWSGGTA |
| 27 | INWTDST |
| 28 | ISWSGSGVVT |
| 29 | IDSGGDRT |
| 30 | ISSRGGTT |
| 31 | VARRTFPSVVEGVDY |
| 32 | STEFTGWDRCGFDEYDH |
| 33 | ARGSHADFNS |
| 34 | AKGTHADFGS |
| 35 | AASTRGIVSDNY |
| 36 | AAGLSLEQHTPPY |
| 37 | ATGIGGLYRRPSAYKY |
| 38 | AARLGTYYKDYARSGAYDT |
| 39 | AKGTHADFGS |
| 40 | AAEFTGWAVCNFSRKSGEYDY |
| 41 | AGMG |
| 42 | YYAIG |
| 43 | SYYMS |
| 44 | SYAMS |
| 45 | LDTMA |
| 46 | RLDMG |
| 47 | RDVMG |
| 48 | SYWLG |
| 49 | SYAMS |
| 50 | YYAID |
| 51 | SISEEGTTTYAVPVEG |
| 52 | CISSSGRTTNYADSVKG |
| 53 | EISSGGGSTNYAVPVEG |
| 54 | EIDSGGGSTDYAVPVKG |
| 55 | STNWNRGGASYADSVKG |
| 56 | SSTWSGGTAYATDSVKT |
| 57 | VINWTDSTYYLDSVKG |
| 58 | AISWSGSGVVTNYADSVKG |
| 59 | EIDSGGDRTDYAVPVKG |
| 60 | CISSRGGTTNYADAVKG |
| 61 | RRTFPSVVEGVDY |
| 62 | EFTGWDRCGFDEYDH |
| 63 | GSHADFNS |
| 64 | GTHADFGS |
| 65 | STRGIVSDNY |
| 66 | GLSLEQHTPPY |
| 67 | GIGGLYRRPSAYKY |
| 68 | RLGTYYKDYARSGAYDT |
| 69 | GTHADFGS |
| 70 | EFTGWAVCNFSRKSGEYDY |
| 71 | EVQLVESGGGLVQPGGSLRLSCAASTRIAGMSWFRQAPGKGRELVASISEEGTTTYADSVKGRFTIS |
| | RDNSTNTLYLQMNSLKPEDTAVYYCVARRTFPSVVEGVDYWGQGTLVTVSS |
| 72 | QVQLVESGGGLIQPGGSLRLSCAASTRIAGMGWYRAPPGKGRELVASISEEGTTTYADSVKGRFTIS |
| | RDNAKNTLYLQMNSLRAEDTAVYYCVARRTFPSVVEGVDYWGQGTQVTVSS |
| 73 | QVQLVESGGGLVQPGGSLRLSCAASTRIAGMSWYRQAPGKGRELVASISEEGTTTYADSVEGRFTI |
| | SRDNAKNTLYLQMNSLRAEDTAVYYCVARRTFPSVVEGVDYWGQGTLVTVSS |
| 74 | QVQLVESGGGLVQPGGSLRLSCAASGDRWDYYAIGWIRQAPGKEREGVSCISSSGRTTNYADSVK |
| | GRFTISRDNAKNTLYLQMNSLRAEDTAVYYCSTEFTGWDRCGFDEYDHRGQGTQVTVSS |
| 75 | QVQLVESGGGLVQPGGSLRLSCAASGDRWDYYAMSWIRQAPGKEREGVSCISSSGRTTNYADSVK |
| | GRFTISRDNARNTLYLQMNSLRAEDTAVYYCSTEFTGWDRCGFDEYDHRGQGTQVTVSS |
| 76 | QVQLVESGGGLVQPGGSLRLSCAASGFTFSSYYMSWVRQPPGKGIEWVSEISSGGGSTNYADSVK |
| | GRFTISRDNAKNTLYLQMNSLRAEDTAVYYCARGSHADFNSWGQGTQVTVSS |
| 77 | QVQLVESGGGLVQPGGSLRLSCAASGFTFSSYYMYWVRQAPGKGLEWVSEISSGGGSTNYADSVK |
| | GRFTISRDNARNTLYLQMNSLRAEDTAVYYCARGSHADFNSWGQGTQVTVSS |
| 78 | QVQLVESGGGLVQPGGSLRLSCAASGFSFSSYAMSWVRQPPGMGIEWVAEIDSGGGSTDYADSVK |
| | GRFTISRDNAKNTLYLQMNSLRAEDTAVYYCAKGTHADFGSWGQGTQVTVSS |
| 79 | EVQLVESGGGLVQPGGSLRLSCAASGFSFSSYAMSWVRQPPGMGIEWVAEIDSGGGSTDYADSVK |
| | GRFTISRDNAKNTLYLQMNSLRAEDTAVYYCAAGTHADFGSWGQGTQVTVSS |
| 80 | EVQLVESGGGLVQPGGSLRLSCAASGFSFSSYAMSWFRQPPGMGRELVAEIDSGGGSTDYADSVK |
| | GRFTISRDNAKNTLYLQMNSLRAEDTAVYYCAKGTHADFGSWGQGTQVTVSS |
| 81 | EVQLVESGGGVVRPGGSLRLSCAASGRTSSLDTMAWFRQAPGKEREFVASTNWNRGGASYADSV |
| | KGRFTISRDNAKNSLYLQMNSLRAEDTALYYCAASTRGIVSDNYWGQGTLVTVSS |
| 82 | EVQLVESGGGVVRPGGSLRLSCAASGRTSSLDTMAWFRQAPGKEREFVASTNWNRGGASYADSV |
| | KGRFTISRDNARNSLYLQMNSLRAEDTALYYCAASTRGIVSDNYWGQGTLVTVSS |
| 83 | QVQLVESGGGLVQPGGSLRLSCAASRGTFSRLDMGWFRQAPGKEREFVASSTWSGGTAYYADSV |
| | KGRFTISRDNAKNTLYLQMNSLRAEDTAVYYCAAGLSLEQHTPPYWGQGTQVTVSS |
| 84 | QVQLVESGGGLVQPGGSLRLSCAASRGTFSRLDMGWFRQAPGKEREFVASSTWSGGTAYYADSV |
| | KGRFTISRDNARNTLYLQMNSLRAEDTAVYYCAAGLSLEQHTPPYWGQGTQVTVSS |
| 85 | EVQLVESGGGLVQPGGSLRLSCAASGRTFSRDVMGWFRQAPGKEREFVAVINWTDSTYYADSVK |
| | GRFTISRDNAKNTLYLQMNSLRAEDTAVYYCATGIGGLYRRPSAYKYWGQGTQVTVSS |
| 86 | EVQLVESGGGLVQPGGSLRLSCAASGRTFSRDVMGWFRQAPGKEREFVAVINWTDSTYYADSVK |
| | GRFTISRDNAENTLYLQMNSLRAEDTAVYYCATGIGGLYRRPSAYRYWGQGTQVTVSS |
| 87 | AVQLVESGGGLVQPGGSLRLSCAASGRTFSSYWLGWFRQVPGEEREFVAAISWSGSGVVTNYADS |
| | VKGRFTISRDNAKNTLYLQMNSLRAEDTAVYYCAARLGTYYKDYARSGAYDTWGQGTQVTVSS |
| 88 | AVQLVESGGGLVQPGGSLRLSCAASGRTFSSYWLGWFRQVPGEEREFVAAISWSGSGVVTNYADS |
| | VKGRFTISRDNAKNTLYLQMNSLRAEDTAVYYCAARLGTYYRDYARSGAYDTWGQGTQVTVSS |
| 89 | QVQLVESGGGLVQPGGSLRLSCAASGFTFSSYAMSWVRQPPGKGIEWVAEIDSGGDRTDYADSVK |
| | GRFTISRDNAKNTLYLQMNSLRAEDTAVYYCAKGTHADFGSWGQGTLVTVSS |
| 90 | EVQLVESGGGLVQPGGSLRLSCAASGFTFSSYAMSWFRQPPGKEREGVAEIDSGGDRTDYADSVK |
| | GRFTISRDNSTNTLYLQMNSLKPEDTAVYYCAAGTHADFGSWGQGTLVTVSS |
| 91 | EVQLVESGGGLVQPGGSLRLSCAASGFTFSSYAMSWFRQPPGKEREGVAEIDSGGDRTDYADSVR |
| | GRFTISRDNSTNTLYLQMNSLKPEDTAVYYCAAGTHADFGSWGQGTLVTVSS |
| 92 | QVQLVESGGGLVQPGESLRLSCAASGFTFSSYAMSWVRQPPGKGIEWVAEIDSGGDRTDYAVPVK |
| | GRFTISRDNVENTLYLQMNSLKPEDTAVFYCAAGTHADFGSWGQGTLVTVSS |
| 93 | QVQLLESGGGLVQPGGSLRLSCAASGFTFSSYAMSWVRQPPGKGIEWVAEIDSGGDRTDYADSVK |
| | GRFTISRDNSRNTLYLQMNSLRAEDTAVYYCAAGTHADFGSWGRGTLVTVSS |
| 94 | QVQLLESGGGLVQPGGSLRLSCAASGFTFSSYAMSWVRQPPGNQIEWVAEIDSGGDRTDYADSVK |
| | GRFTISRDNSKNTLYLQMNSLRAEDTAVYYCAAGTHADFGSWGRGTLVTVSS |
| 95 | QVQLVESGGGLVQPGGSLRLSCTASGDTLDYYAIDWFRQAPGKEREGVSCISSRGGTTNYADSVK |
| | GRFTISRDNAKNTLYLQMNSLRAEDTAVYYCAAEFTGWAVCNFSRKSGEYDYWGQGTQVTVSS |
| 96 | EVQLVESGGGLVQPGGSLRLSCTASGDTLDYYAMHWFRQAPGKEREGVSCISSRGGTTNYADSVK |
| | GRFTISRDNAKNTLYLQMNSLRAEDTAVYYCAAEFTGWAVCNFSRKSGEYDYWGQGTQVTVSS |
| 97 | QVQLLESGGGLVQPGGSLRLSCAASGDTLDYYAMSWFRQAPGKEREGVSCISSRGGTTNYADSVK |
| | GRFTISRDNARNTLYLQMNSLRAEDTAVYYCAAEFTGWAVCNFSRRSGEYDYWGQGTQVTVSS |
| 98 | KLTIESTPFNVAEGKEVLLLVHNLPQHLFGYSWYKGERVDGNRQIIGYVIGTQQATPGPAYSGREII |
| | YPNASLLIQNIIQNDTGFYTLHVIKSDLVNEEATGQFRVYPELPKPSISSNNSKPVEDKDAVAFTCE |
| | PETQDATYLWWVNNQSLPVSPRLQLSNGNRTLTLFNVTRNDTASYKCETQNPVSARRSDSVILNVL |
| | YGPDAPTISPLNTSYRSGENLNLSCHAASNPPAQYSWFVNGTFQQSTQELFIPNITVNNSGSYTCQA |
| | HNSDTGLNRTTVTTITVYAEPPKPFITSNNSNPVEDEDAVALTCEPEIQNTTYLWWVNNQSLPVSPR |
| | LQLSNDNRTLTLLSVTRNDVGPYECGIQNELSVDHSDPVILNVLYGPDDPTISPSYTYYRPGVNLSL |
| | SCHAASNPPAQYSWLIDGNIQQHTQELFISNITEKNSGLYTCQANNSASGHSRTTVKTITVSAELPK |
| | PSISSNNSKPVEDKDAVAFTCEPEAQNTTYLWWVNGQSLPVSPRLQLSNGNRTLTLFNVTRNDARAY |
| | VCGIQNSVSANRSDPVTLDVLYGPDTPIISPPDSSYLSGANLNLSCHSASNPSPQYSWRINGIPQQH |
| | TQVLFIAKITPNNNGTYACFVSNLATGRNNSIVKSITVSASGTSPGLSA |
| 99 | ELPKPSISSNNSKPVEDKDAVAFTCEPEAQNTTYLWWVNGQSLPVSPRLQLSNGNRTLTLFNVTRN |
| | DARAYVCGIQNSVSANRSDPVTLDVLYGPDTPIISPPDSSYLSGANLNLSCHSASNPSPQYSWRIN |
| | PQQHTQVLFIAKITPNNNGTYACFVSNLATGRNNSIVKSITVSASGTSPGLSA |
| 100 | GAAGTGCAGCTGGTGGAGAGCGGAGGAGGATTGGTGCAGGCAGGAGGAAGCCTGAGGCTGAG |
| | TTGTGTGGCTAGTACAAGGATCGCCGGAATGGGATGGTATAGGGCCCCTCCAGGGAAGGGTAG |
| | GGAGTTGGTTGCCAGCATCAGCGAGGAGGGAACAACCACCTACGCAGTGCCCGTGGAGGGTA |
| | GATTCACCGTGAGCAGGGATAACGTGAAGAACATGCTGCATCTGCAGATGAATAGCCTGAAGC |
| | CCGAGGACACCGCCATGTACTATTGCGTGGCCAGGAGGACCTTCCCCAGCGTTGTTGAGGGCG |
| | TGGACTACTGGGGCCAGGGAACACAGGTGACCGTGAGTAGC |
| 101 | CAGTTGCAGCTCGTGGAGTCCGGTGGAGGACTCGTTCAGCCTGGAGGATCACTCAGGCTCTCC |
| | TGCGCAGTCTCCGGTGATAGATGGGACTATTATGCTATTGGCTGGATCAGGCAGGCCCCCGGA |
| | AAAGAAAGGGAGGGTGTGAGCTGCATTTCCTCTAGTGGGCGGACCACCAACTACGCCGATAGC |
| | GTTAAGGGCAGGTTCACAATCTCCAGGGACAATGCTAAGAACACCGTTTACCTGCAGATGAAC |
| | AGCTTGAAGCCCGAGGACACCGCCGTGTATTACTGCTCCACCGAGTTTACCGGATGGGACAGG |
| | TGCGGATTCGACGAGTACGATCACCGTGGACAGGGCACCCAGGTGACAGTTAGCTCC |
| 102 | GAGGTTGACTTGGTGGAGAGCGGAGGAGGATTGGTGCAGCCTGGAGGAAGTTTGAGGTTGAG |
| | CTGTGCCGCATCCGGGTTTACCTTTAGTTCCTATTACATGTCCTGGGTGAGGCAGCCACCTGGA |
| | AAGGGAATTGAGTGGGTGAGCGAGATTAGTAGCGGAGGAGGAAGCACAAACTACGCCGTTCC |
| | CGTCGAGGGAAGATTCACCATCTCCAGGGACAACGCTAAGAACACACTCTCCCTGCAGATGAA |
| | CAGCCTGAAGCCAGAGGACACAGCCCTGTATTACTGCGCCAGAGGCAGCCATGCCGATTTTAA |
| | CAGCTGGGGCCAGGGCACACAGGTGACCGTGTCCTCC |
| 103 | GAAGTGCAGCTGGTGGAGAGCGGAGGAGGATTGGTGCAGCCTGGAGAGAGCCTGAGGCTGAG |
| | TTGTGCCGCAAGCGGATTTAGTTTTTCCTCCTACGCCATGTCCTGGGTGAGGCAGCCTCCTGGA |
| | ATGGGAATCGAATGGGTGGCCGAGATTGACAGCGGAGGAGGAAGCACCGACTACGCTGTTCC |
| | CGTGAAGGGCAGGTTTACCATTTCCCGGGACAACGTTAAGAACACCCTGTATCTGCAGATGAA |
| | CAGCCTGAAGCCTGAGGACACCGCCGTGTTTTATTGCGCCAAGGGCACCCACGCCGATTTCGG |
| | ATCTTGGGGACAGGGCACACAGGTGACCGTGAGTTCC |
| 104 | GAAGTGGACCTGGTGGAGAGCGGAGGAGGATTGGTGCAGGCAGGAGGAAGCCTGAGGCTGAG |
| | TTGTGCCCCTAGCGGAAGGACAAGTAGTCTGGATACAATGGCATGGTTCAGGCAGGCCCCCGG |
| | AAAGGAAAGGGAGTTCGTGGCTTCTACCAACTGGAATAGAGGAGGAGCATCTTACGCCGATTC |
| | CGTGAAGGGGAGGTTCACCATTTCCAGGGACGCCACTAAGAACACAGTCTACCTGCAGATCAA |
| | CAGCCTGGAGCCTGAGGACACCGCCGTTTACTATTGCGCCGCCTCCACCAGGGGCATCGTTTCT |
| | GATAACTACTGGGGCCAGGGAACCCAGGTGACCGTTTCTTCC |
| 105 | GAAGTGCAGCTGGTGGAAAGCGGAGGAGGATTGGTGCAGCCTGGAGGAAGTCTGAGGCTGAG |
| | TTGTGCAGCAAGCAGGGGAACATTTAGCAGGCTGGACATGGGATGGTTTAGGCAGGCCCCTGG |
| | AAAGGAAAGGGAGTTCGTGGCTAGTTCCACCTGGAGCGGAGGAACAGCATACGCAACCGACA |
| | GTGTGAAAACTCGTTTTACAATCTCTAGGGACAGCGCCAAGACAACAGTGTACCTGCAGATGA |
| | ACAGCCTGAGGCCCGAGGATACCGCTGTTTATTATTGCGCCGCCGGACTGAGCCTGGAACAGC |
| | ATACACCTCCCTACTGGGGCCAGGGAACACAGGTTACAGTGTCCTCT |
| 106 | GAGGTTCAGTTGGTGGAGTCCGGAGGAGGATTGGTGCAGGCTGGAGGAAGTCTGAGGTTGTCC |
| | TGTGCCGCTAGTGGAAGGACCTTTAGTAGGGACGTGATGGGGTGGTTTAGGCAGGCTCCTGGA |
| | AAGGAGAGGGAGTTCGTGGCTGTGATTAACTGGACTGACTCCACCTACTACCTGGATTCCGTT |
| | AAGGGGAGGTTCACCATTTCAAGGGAGAACGCTGAGAACACCGTGTATCTGCAGATGAACAG |
| | CCTGAAGCCTGAAGATACTGCTATTTACTACTGCGCCACCGGCATTGGGGGCCTGTACAGGAG |
| | GCCTTCCGCTTACAAGTATTGGGGACAGGGCACACAGGTCACCGTTAGTAGC |
| 107 | GCTGTTCAGTTGGTGGAGTCCGGGGGAGGATTGGTGCAGGCTGGAGGATCTCTGAGGTTGTCT |
| | TGCGCCGCTTCTGGGAGGACTTTTTCTTCTTATTGGTTGGGGTGGTTTAGGCAGGTGCCTGGGG |
| | AGGAAAGGGAGTTTGTGGCTGCTATTTCTTGGAGTGGATCTGGCGTGGTGACTAATTATGCTG |
| | ACAGTGTGAAGGGCCGGTTTACTATTAGCAGGGACGACGGAAACAACGCCTTGTACCTGCAGA |
| | TGTCCAATCTGAACCCCGAGGACACCGCTGTTTATTATTGCGCCGCCCGGCTGGGAACCTATTA |
| | CAAGGACTACGCCCGGTCCGGGGCCTATGATACATGGGGACAGGGCACCCAGGTGACCGTTTC |
| | TTCC |
| 108 | CAGGTTCAGCTCGTCGAGTCAGGCGGAGGACTCGTTCAGCCAGGAGAAAGCCTCAGGCTGTCA |
| | TGCGCCGCTTCCGGTTTTACATTCTCCTCCTACGCCATGTCCTGGGTGAGGCAGCCACCTGGAA |
| | AGGGTATTGAGTGGGTGGCTGAGATTGACAGCGGAGGAGACAGGACAGACTACGCCGTGCCT |
| | GTGAAGGGCAGGTTTACCATCTCCAGAGACAACGTGGAGAACACACTCTATCTGCAGATGAAC |
| | AGCTTGAAGCCCGAAGATACTGCTGTGTTTTACTGCGCCAAAGGCACACACGCCGATTTCGGA |
| | AGCTGGGGACAGGGAACCCTGGTGACAGTGTCCTCC |
| 109 | GAGGTTGACTTGATGGAGAGCGGGGGAGGATTGGTGCAGCCTGGAGGAAGCCTGAGATTGAG |
| | CTGTACCGCTAGTGGAGACACACTGGACTACTACGCTATCGACTGGTTCAGGCAGGCACCTGG |
| | AAAGGAGAGGGAGGGAGTTAGCTGCATTTCCAGCAGGGGAGGAACCACCAACTACGCTGACG |
| | CTGTGAAGGGAAGATTCACTATCAGCAGGGACAACGCCAAGAACACAGTGTACCTGCAGATG |
| | AACAGCCTGAGGCCCGAGGATACCGCAGTTTACTACTGCGCCGCCGAGTTCACAGGATGGGCT |
| | GTGTGCAACTTCTCCAGGAAGAGCGGAGAGTACGACTACTGGGGCCAGGGAACACAGGTGAC |
| | AGTGAGTAGC |

1: An antibody specifically binding to carcinoembryonic antigen-related cell adhesion molecule 5 (CEACAM5) (anti-CEACAM5 antibody), comprising CDR1, CDR2, and CDR3 of a single domain antibody comprising an amino acid sequence selected from the group consisting of SEQ ID NOs: 1-10.

2: The anti-CEACAM5 antibody of claim 1, wherein the CDR1, CDR2 and CDR3 are according to IMGT numbering or Kabat numbering.

3. (canceled)

4: An antibody specifically binding to carcinoembryonic antigen-related cell adhesion molecule 5 (CEACAM5) (anti-CEACAM5 antibody), wherein the antibody comprises:
(1) a CDR 1 comprising the amino acid sequence of SEQ ID NO: 11, a CDR2 comprising the amino acid sequence of SEQ ID NO: 21, and a CDR3 comprising the amino acid sequence of SEQ ID NO: 31;
(2) a CDR1 comprising the amino acid sequence of SEQ ID NO: 12, a CDR2 comprising the amino acid sequence of SEQ ID NO: 22, and a CDR3 comprising the amino acid sequence of SEQ ID NO: 32;
(3) a CDR1 comprising the amino acid sequence of SEQ ID NO: 13, a CDR2 comprising the amino acid sequence of SEQ ID NO: 23, and a CDR3 comprising the amino acid sequence of SEQ ID NO: 33;
(4) a CDR1 comprising the amino acid sequence of SEQ ID NO: 14, a CDR2 comprising the amino acid sequence of SEQ ID NO: 24, and a CDR3 comprising the amino acid sequence of SEQ ID NO: 34;
(5) a CDR1 comprising the amino acid sequence of SEQ ID NO: 15, a CDR2 comprising the amino acid sequence of SEQ ID NO: 25, and a CDR3 comprising the amino acid sequence of SEQ ID NO: 35;
(6) a CDR1 comprising the amino acid sequence of SEQ ID NO: 16, a CDR2 comprising the amino acid sequence of SEQ ID NO: 26, and a CDR3 comprising the amino acid sequence of SEQ ID NO: 36;
(7) a CDR1 comprising the amino acid sequence of SEQ ID NO: 17, a CDR2 comprising the amino acid sequence of SEQ ID NO: 27, and a CDR3 comprising the amino acid sequence of SEQ ID NO: 37;
(8) a CDR1 comprising the amino acid sequence of SEQ ID NO: 18, a CDR2 comprising the amino acid sequence of SEQ ID NO: 28, and a CDR3 comprising the amino acid sequence of SEQ ID NO: 38;
(9) a CDR1 comprising the amino acid sequence of SEQ ID NO: 19, a CDR2 comprising the amino acid sequence of SEQ ID NO: 29, and a CDR3 comprising the amino acid sequence of SEQ ID NO: 39; or
(10) a CDR1 comprising the amino acid sequence of SEQ ID NO: 20, a CDR2 comprising the amino acid sequence of SEQ ID NO: 30, and a CDR3 comprising the amino acid sequence of SEQ ID NO: 40.

5: The anti-CEACAM5 antibody of claim 4, wherein the anti-CEACAM5 antibody comprises:
a) an amino acid sequence having at least about 70% identity with an amino acid sequence selected from the group consisting of SEQ ID NOs: 1-10 and 71-97; or
b) an amino acid sequence selected from the group consisting of SEQ ID NOs: 1-10 and 71-97.

6: The anti-CEACAM5 antibody of claim 4, wherein the anti-CEACAM5 antibody is a single domain antibody.

7: An anti-CEACAM5 antibody construct, comprising the anti-CEACAM5 antibody of claim 6.

8: The anti-CEACAM5 antibody construct of claim 7, further comprising an Fc fragment.

9: The anti-CEACAM5 antibody construct of claim 8, wherein the Fc fragment is derived from any of IgG1, IgG2, IgG3 and IgG4.

10: The anti-CEACAM5 antibody construct of claim 7, wherein the anti-CEACAM5 antibody construct is multi-specific.

11: The anti-CEACAM5 antibody construct of claim 10, wherein the anti-CEACAM5 antibody construct is bispecific.

12: An isolated nucleic acid encoding the anti-CEACAM5 antibody of claim 6.

13-14. (canceled)

15: A vector comprising the isolated nucleic acid of claim 12.

16: A host cell comprising the nucleic acid of claim 12 or a vector comprising thereof.

17: A conjugate comprising:
(1) the anti-CEACAM5 antibody of claim 4, and
(2) a drug.

18. (canceled)

19: A chimeric antigen receptor (CAR) comprising the anti-CEACAM5 antibody construct of claim 7.

20: An engineered T-cell receptor (TCR) comprising the anti-CEACAM5 antibody construct of claim 7.

21: A cell expressing the CAR of claim 19.

22: A pharmaceutical composition, comprising:
(1) the anti-CEACAM5 antibody of claim 4, and
(2) a pharmaceutically acceptable excipient.

23: A method for treating a disorder in a subject in need thereof, comprising administering an effective amount of the pharmaceutical composition of claim 22 to the subject.

24-26. (canceled)

27: A method of detecting CEACAM5 in a sample from a subject, comprising contacting the sample with the anti-CEACAM5 antibody of claim 4, wherein detection of the anti-CEACAM5 antibody indicates presence of CEACAM5 in the sample.

28: A method of preparing the anti-CEACAM5 antibody of claim 4, comprising: (a) culturing the host cell of claim 16, under a condition effective to express the encoded antibody or antibody construct; and (b) obtaining the expressed antibody or antibody construct from the host cell.

29: A cell expressing the engineered TCR of claim 20.


SEQUENCE LISTING (109 sequences)

SEQ ID NO: 1 - protein, 118 aa, synthetic construct
EVQLVESGGGLVQAGGSLRLSCVASTRIAGMGWYRAPPGKGRELVASISEEGTTTYAVPVEGRFTVSRDNVKNMLHLQMNSLKPEDTAMYYCVARRTFPSVVEGVDYWGQGTQVTVSS

SEQ ID NO: 2 - protein, 124 aa, synthetic construct
QLQLVESGGGLVQPGGSLRLSCAVSGDRWDYYAIGWIRQAPGKEREGVSCISSSGRTTNYADSVKGRFTISRDNAKNTVYLQMNSLKPEDTAVYYCSTEFTGWDRCGFDEYDHRGQGTQVTVSS

SEQ ID NO: 3 - protein, 117 aa, synthetic construct
EVDLVESGGGLVQPGGSLRLSCAASGFTFSSYYMSWVRQPPGKGIEWVSEISSGGGSTNYAVPVEGRFTISRDNAKNTLSLQMNSLKPEDTALYYCARGSHADFNSWGQGTQVTVSS

SEQ ID NO: 4 - protein, 117 aa, synthetic construct
EVQLVESGGGLVQPGESLRLSCAASGFSFSSYAMSWVRQPPGMGIEWVAEIDSGGGSTDYAVPVKGRFTISRDNVKNTLYLQMNSLKPEDTAVFYCAKGTHADFGSWGQGTQVTVSS

SEQ ID NO: 5 - protein, 119 aa, synthetic construct
EVDLVESGGGLVQAGGSLRLSCAPSGRTSSLDTMAWFRQAPGKEREFVASTNWNRGGASYADSVKGRFTISRDATKNTVYLQINSLEPEDTAVYYCAASTRGIVSDNYWGQGTQVTVSS

SEQ ID NO: 6 - protein, 120 aa, synthetic construct
EVQLVESGGGLVQPGGSLRLSCAASRGTFSRLDMGWFRQAPGKEREFVASSTWSGGTAYATDSVKTRFTISRDSAKTTVYLQMNSLRPEDTAVYYCAAGLSLEQHTPPYWGQGTQVTVSS

SEQ ID NO: 7 - protein, 122 aa, synthetic construct
EVQLVESGGGLVQAGGSLRLSCAASGRTFSRDVMGWFRQAPGKEREFVAVINWTDSTYYLDSVKGRFTISRENAENTVYLQMNSLKPEDTAIYYCATGIGGLYRRPSAYKYWGQGTQVTVSS

SEQ ID NO: 8 - protein, 128 aa, synthetic construct
AVQLVESGGGLVQAGGSLRLSCAASGRTFSSYWLGWFRQVPGEEREFVAAISWSGSGVVTNYADSVKGRFTISRDDGNNALYLQMSNLNPEDTAVYYCAARLGTYYKDYARSGAYDTWGQGTQVTVSS

SEQ ID NO: 9 - protein, 117 aa, synthetic construct
QVQLVESGGGLVQPGESLRLSCAASGFTFSSYAMSWVRQPPGKGIEWVAEIDSGGDRTDYAVPVKGRFTISRDNVENTLYLQMNSLKPEDTAVFYCAKGTHADFGSWGQGTLVTVSS

SEQ ID NO: 10 - protein, 128 aa, synthetic construct
EVDLMESGGGLVQPGGSLRLSCTASGDTLDYYAIDWFRQAPGKEREGVSCISSRGGTTNYADAVKGRFTISRDNAKNTVYLQMNSLRPEDTAVYYCAAEFTGWAVCNFSRKSGEYDYWGQGTQVTVSS

SEQ ID NO: 11 - protein, 5 aa, synthetic construct
TRIAG

SEQ ID NO: 12 - protein, 8 aa, synthetic construct
GDRWDYYA

SEQ ID NO: 13 - protein, 8 aa, synthetic construct
GFTFSSYY

SEQ ID NO: 14 - protein, 8 aa, synthetic construct
GFSFSSYA

SEQ ID NO: 15 - protein, 8 aa, synthetic construct
GRTSSLDT

SEQ ID NO: 16 - protein, 8 aa, synthetic construct
RGTFSRLD

SEQ ID NO: 17 - protein, 8 aa, synthetic construct
GRTFSRDV

SEQ ID NO: 18 - protein, 8 aa, synthetic construct
GRTFSSYW

SEQ ID NO: 19 - protein, 8 aa, synthetic construct
GFTFSSYA

SEQ ID NO: 20 - protein, 8 aa, synthetic construct
GDTLDYYA

SEQ ID NO: 21 - protein, 7 aa, synthetic construct
ISEEGTT

SEQ ID NO: 22 - protein, 8 aa, synthetic construct
ISSSGRTT

SEQ ID NO: 23 - protein, 8 aa, synthetic construct
ISSGGGST

SEQ ID NO: 24 - protein, 8 aa, synthetic construct
IDSGGGST

SEQ ID NO: 25 - protein, 8 aa, synthetic construct
TNWNRGGA

SEQ ID NO: 26 - protein, 8 aa, synthetic construct
STWSGGTA

SEQ ID NO: 27 - protein, 7 aa, synthetic construct
INWTDST

SEQ ID NO: 28 - protein, 10 aa, synthetic construct
ISWSGSGVVT

SEQ ID NO: 29 - protein, 8 aa, synthetic construct
IDSGGDRT

SEQ ID NO: 30 - protein, 8 aa, synthetic construct
ISSRGGTT

SEQ ID NO: 31 - protein, 15 aa, synthetic construct
VARRTFPSVVEGVDY

SEQ ID NO: 32 - protein, 17 aa, synthetic construct
STEFTGWDRCGFDEYDH

SEQ ID NO: 33 - protein, 10 aa, synthetic construct
ARGSHADFNS

SEQ ID NO: 34 - protein, 10 aa, synthetic construct
AKGTHADFGS

SEQ ID NO: 35 - protein, 12 aa, synthetic construct
AASTRGIVSDNY

SEQ ID NO: 36 - protein, 13 aa, synthetic construct
AAGLSLEQHTPPY

SEQ ID NO: 37 - protein, 16 aa, synthetic construct
ATGIGGLYRRPSAYKY

SEQ ID NO: 38 - protein, 19 aa, synthetic construct
AARLGTYYKDYARSGAYDT

SEQ ID NO: 39 - protein, 10 aa, synthetic construct
AKGTHADFGS

SEQ ID NO: 40 - protein, 21 aa, synthetic construct
AAEFTGWAVCNFSRKSGEYDY

SEQ ID NO: 41 - protein, 4 aa, synthetic construct
AGMG

SEQ ID NO: 42 - protein, 5 aa, synthetic construct
YYAIG

SEQ ID NO: 43 - protein, 5 aa, synthetic construct
SYYMS

SEQ ID NO: 44 - protein, 5 aa, synthetic construct
SYAMS

SEQ ID NO: 45 - protein, 5 aa, synthetic construct
LDTMA

SEQ ID NO: 46 - protein, 5 aa, synthetic construct
RLDMG

SEQ ID NO: 47 - protein, 5 aa, synthetic construct
RDVMG

SEQ ID NO: 48 - protein, 5 aa, synthetic construct
SYWLG

SEQ ID NO: 49 - protein, 5 aa, synthetic construct
SYAMS

SEQ ID NO: 50 - protein, 5 aa, synthetic construct
YYAID

SEQ ID NO: 51 - protein, 16 aa, synthetic construct
SISEEGTTTYAVPVEG

SEQ ID NO: 52 - protein, 17 aa, synthetic construct
CISSSGRTTNYADSVKG

SEQ ID NO: 53 - protein, 17 aa, synthetic construct
EISSGGGSTNYAVPVEG

SEQ ID NO: 54 - protein, 17 aa, synthetic construct
EIDSGGGSTDYAVPVKG

SEQ ID NO: 55 - protein, 17 aa, synthetic construct
STNWNRGGASYADSVKG

SEQ ID NO: 56 - protein, 17 aa, synthetic construct
SSTWSGGTAYATDSVKT

SEQ ID NO: 57 - protein, 16 aa, synthetic construct
VINWTDSTYYLDSVKG

SEQ ID NO: 58 - protein, 19 aa, synthetic construct
AISWSGSGVVTNYADSVKG

SEQ ID NO: 59 - protein, 17 aa, synthetic construct
EIDSGGDRTDYAVPVKG

SEQ ID NO: 60 - protein, 17 aa, synthetic construct
CISSRGGTTNYADAVKG

SEQ ID NO: 61 - protein, 13 aa, synthetic construct
RRTFPSVVEGVDY

SEQ ID NO: 62 - protein, 15 aa, synthetic construct
EFTGWDRCGFDEYDH

SEQ ID NO: 63 - protein, 8 aa, synthetic construct
GSHADFNS

SEQ ID NO: 64 - protein, 8 aa, synthetic construct
GTHADFGS

SEQ ID NO: 65 - protein, 10 aa, synthetic construct
STRGIVSDNY

SEQ ID NO: 66 - protein, 11 aa, synthetic construct
GLSLEQHTPPY

SEQ ID NO: 67 - protein, 14 aa, synthetic construct
GIGGLYRRPSAYKY

SEQ ID NO: 68 - protein, 17 aa, synthetic construct
RLGTYYKDYARSGAYDT

SEQ ID NO: 69 - protein, 8 aa, synthetic construct
GTHADFGS

SEQ ID NO: 70 - protein, 19 aa, synthetic construct
EFTGWAVCNFSRKSGEYDY

SEQ ID NO: 71 - protein, 118 aa, synthetic construct
EVQLVESGGGLVQPGGSLRLSCAASTRIAGMSWFRQAPGKGRELVASISEEGTTTYADSVKGRFTISRDNSTNTLYLQMNSLKPEDTAVYYCVARRTFPSVVEGVDYWGQGTLVTVSS

SEQ ID NO: 72 - protein, 118 aa, synthetic construct
QVQLVESGGGLIQPGGSLRLSCAASTRIAGMGWYRAPPGKGRELVASISEEGTTTYADSVKGRFTISRDNAKNTLYLQMNSLRAEDTAVYYCVARRTFPSVVEGVDYWGQGTQVTVSS

SEQ ID NO: 73 - protein, 118 aa, synthetic construct
QVQLVESGGGLVQPGGSLRLSCAASTRIAGMSWYRQAPGKGRELVASISEEGTTTYADSVEGRFTISRDNAKNTLYLQMNSLRAEDTAVYYCVARRTFPSVVEGVDYWGQGTLVTVSS

SEQ ID NO: 74 - protein, 124 aa, synthetic construct
QVQLVESGGGLVQPGGSLRLSCAASGDRWDYYAIGWIRQAPGKEREGVSCISSSGRTTNYADSVKGRFTISRDNAKNTLYLQMNSLRAEDTAVYYCSTEFTGWDRCGFDEYDHRGQGTQVTVSS

SEQ ID NO: 75 - protein, 124 aa, synthetic construct
QVQLVESGGGLVQPGGSLRLSCAASGDRWDYYAMSWIRQAPGKEREGVSCISSSGRTTNYADSVKGRFTISRDNARNTLYLQMNSLRAEDTAVYYCSTEFTGWDRCGFDEYDHRGQGTQVTVSS

SEQ ID NO: 76 - protein, 117 aa, synthetic construct
QVQLVESGGGLVQPGGSLRLSCAASGFTFSSYYMSWVRQPPGKGIEWVSEISSGGGSTNYADSVKGRFTISRDNAKNTLYLQMNSLRAEDTAVYYCARGSHADFNSWGQGTQVTVSS

SEQ ID NO: 77 - protein, 117 aa, synthetic construct
QVQLVESGGGLVQPGGSLRLSCAASGFTFSSYYMYWVRQAPGKGLEWVSEISSGGGSTNYADSVKGRFTISRDNARNTLYLQMNSLRAEDTAVYYCARGSHADFNSWGQGTQVTVSS

SEQ ID NO: 78 - protein, 117 aa, synthetic construct
QVQLVESGGGLVQPGGSLRLSCAASGFSFSSYAMSWVRQPPGMGIEWVAEIDSGGGSTDYADSVKGRFTISRDNAKNTLYLQMNSLRAEDTAVYYCAKGTHADFGSWGQGTQVTVSS

SEQ ID NO: 79 - protein, 117 aa, synthetic construct
EVQLVESGGGLVQPGGSLRLSCAASGFSFSSYAMSWVRQPPGMGIEWVAEIDSGGGSTDYADSVKGRFTISRDNAKNTLYLQMNSLRAEDTAVYYCAAGTHADFGSWGQGTQVTVSS

SEQ ID NO: 80 - protein, 117 aa, synthetic construct
EVQLVESGGGLVQPGGSLRLSCAASGFSFSSYAMSWFRQPPGMGRELVAEIDSGGGSTDYADSVKGRFTISRDNAKNTLYLQMNSLRAEDTAVYYCAKGTHADFGSWGQGTQVTVSS

SEQ ID NO: 81 - protein, 119 aa, synthetic construct
EVQLVESGGGVVRPGGSLRLSCAASGRTSSLDTMAWFRQAPGKEREFVASTNWNRGGASYADSVKGRFTISRDNAKNSLYLQMNSLRAEDTALYYCAASTRGIVSDNYWGQGTLVTVSS

SEQ ID NO: 82 - protein, 119 aa, synthetic construct
EVQLVESGGGVVRPGGSLRLSCAASGRTSSLDTMAWFRQAPGKEREFVASTNWNRGGASYADSVKGRFTISRDNARNSLYLQMNSLRAEDTALYYCAASTRGIVSDNYWGQGTLVTVSS

SEQ ID NO: 83 - protein, 120 aa, synthetic construct
QVQLVESGGGLVQPGGSLRLSCAASRGTFSRLDMGWFRQAPGKEREFVASSTWSGGTAYYADSVKGRFTISRDNAKNTLYLQMNSLRAEDTAVYYCAAGLSLEQHTPPYWGQGTQVTVSS

SEQ ID NO: 84 - protein, 120 aa, synthetic construct
QVQLVESGGGLVQPGGSLRLSCAASRGTFSRLDMGWFRQAPGKEREFVASSTWSGGTAYYADSVKGRFTISRDNARNTLYLQMNSLRAEDTAVYYCAAGLSLEQHTPPYWGQGTQVTVSS

SEQ ID NO: 85 - protein, 122 aa, synthetic construct
EVQLVESGGGLVQPGGSLRLSCAASGRTFSRDVMGWFRQAPGKEREFVAVINWTDSTYYADSVKGRFTISRDNAKNTLYLQMNSLRAEDTAVYYCATGIGGLYRRPSAYKYWGQGTQVTVSS

SEQ ID NO: 86 - protein, 122 aa, synthetic construct
EVQLVESGGGLVQPGGSLRLSCAASGRTFSRDVMGWFRQAPGKEREFVAVINWTDSTYYADSVKGRFTISRDNAENTLYLQMNSLRAEDTAVYYCATGIGGLYRRPSAYRYWGQGTQVTVSS

SEQ ID NO: 87 - protein, 128 aa, synthetic construct
AVQLVESGGGLVQPGGSLRLSCAASGRTFSSYWLGWFRQVPGEEREFVAAISWSGSGVVTNYADSVKGRFTISRDNAKNTLYLQMNSLRAEDTAVYYCAARLGTYYKDYARSGAYDTWGQGTQVTVSS

SEQ ID NO: 88 - protein, 128 aa, synthetic construct
AVQLVESGGGLVQPGGSLRLSCAASGRTFSSYWLGWFRQVPGEEREFVAAISWSGSGVVTNYADSVKGRFTISRDNAKNTLYLQMNSLRAEDTAVYYCAARLGTYYRDYARSGAYDTWGQGTQVTVSS

SEQ ID NO: 89 - protein, 117 aa, synthetic construct
QVQLVESGGGLVQPGGSLRLSCAASGFTFSSYAMSWVRQPPGKGIEWVAEIDSGGDRTDYADSVKGRFTISRDNAKNTLYLQMNSLRAEDTAVYYCAKGTHADFGSWGQGTLVTVSS

SEQ ID NO: 90 - protein, 117 aa, synthetic construct
EVQLVESGGGLVQPGGSLRLSCAASGFTFSSYAMSWFRQPPGKEREGVAEIDSGGDRTDYADSVKGRFTISRDNSTNTLYLQMNSLKPEDTAVYYCAAGTHADFGSWGQGTLVTVSS

SEQ ID NO: 91 - protein, 117 aa, synthetic construct
EVQLVESGGGLVQPGGSLRLSCAASGFTFSSYAMSWFRQPPGKEREGVAEIDSGGDRTDYADSVRGRFTISRDNSTNTLYLQMNSLKPEDTAVYYCAAGTHADFGSWGQGTLVTVSS

SEQ ID NO: 92 - protein, 117 aa, synthetic construct
QVQLVESGGGLVQPGESLRLSCAASGFTFSSYAMSWVRQPPGKGIEWVAEIDSGGDRTDYAVPVKGRFTISRDNVENTLYLQMNSLKPEDTAVFYCAAGTHADFGSWGQGTLVTVSS

SEQ ID NO: 93 - protein, 117 aa, synthetic construct
QVQLLESGGGLVQPGGSLRLSCAASGFTFSSYAMSWVRQPPGKGIEWVAEIDSGGDRTDYADSVKGRFTISRDNSRNTLYLQMNSLRAEDTAVYYCAAGTHADFGSWGRGTLVTVSS

SEQ ID NO: 94 - protein, 117 aa, synthetic construct
QVQLLESGGGLVQPGGSLRLSCAASGFTFSSYAMSWVRQPPGNQIEWVAEIDSGGDRTDYADSVKGRFTISRDNSKNTLYLQMNSLRAEDTAVYYCAAGTHADFGSWGRGTLVTVSS

SEQ ID NO: 95 - protein, 128 aa, synthetic construct
QVQLVESGGGLVQPGGSLRLSCTASGDTLDYYAIDWFRQAPGKEREGVSCISSRGGTTNYADSVKGRFTISRDNAKNTLYLQMNSLRAEDTAVYYCAAEFTGWAVCNFSRKSGEYDYWGQGTQVTVSS

SEQ ID NO: 96 - protein, 128 aa, synthetic construct
EVQLVESGGGLVQPGGSLRLSCTASGDTLDYYAMHWFRQAPGKEREGVSCISSRGGTTNYADSVKGRFTISRDNAKNTLYLQMNSLRAEDTAVYYCAAEFTGWAVCNFSRKSGEYDYWGQGTQVTVSS

SEQ ID NO: 97 - protein, 128 aa, synthetic construct
QVQLLESGGGLVQPGGSLRLSCAASGDTLDYYAMSWFRQAPGKEREGVSCISSRGGTTNYADSVKGRFTISRDNARNTLYLQMNSLRAEDTAVYYCAAEFTGWAVCNFSRRSGEYDYWGQGTQVTVSS

SEQ ID NO: 98 - protein, 651 aa, synthetic construct
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 99 - protein, 187 aa, synthetic construct
ELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 100 - DNA, 354 bp, synthetic construct
  source 1..354: mol_type = other DNA
gaagtgcagctggtggagagcggaggaggattggtgcaggcaggaggaagcctgaggctgagttgtgtggctagtacaaggatcgccggaatgggatggtatagggcccctccagggaagggtagggagttggttgccagcatcagcgaggagggaacaaccacctacgcagtgcccgtggagggtagattcaccgtgagcagggataacgtgaagaacatgctgcatctgcagatgaatagcctgaagcccgaggacaccgccatgtactattgcgtggccaggaggaccttccccagcgttgttgagggcgtggactactggggccagggaacacaggtgaccgtgagtagc

SEQ ID NO: 101 - DNA, 372 bp, synthetic construct
  source 1..372: mol_type = other DNA
cagttgcagctcgtggagtccggtggaggactcgttcagcctggaggatcactcaggctctcctgcgcagtctccggtgatagatgggactattatgctattggctggatcaggcaggcccccggaaaagaaagggagggtgtgagctgcatttcctctagtgggcggaccaccaactacgccgatagcgttaagggcaggttcacaatctccagggacaatgctaagaacaccgtttacctgcagatgaacagcttgaagcccgaggacaccgccgtgtattactgctccaccgagtttaccggatgggacaggtgcggattcgacgagtacgatcaccgtggacagggcacccaggtgacagttagctcc

SEQ ID NO: 102 - DNA, 351 bp, synthetic construct
  source 1..351: mol_type = other DNA
gaggttgacttggtggagagcggaggaggattggtgcagcctggaggaagtttgaggttgagctgtgccgcatccgggtttacctttagttcctattacatgtcctgggtgaggcagccacctggaaagggaattgagtgggtgagcgagattagtagcggaggaggaagcacaaactacgccgttcccgtcgagggaagattcaccatctccagggacaacgctaagaacacactctccctgcagatgaacagcctgaagccagaggacacagccctgtattactgcgccagaggcagccatgccgattttaacagctggggccagggcacacaggtgaccgtgtcctcc

SEQ ID NO: 103 - DNA, 351 bp, synthetic construct
  source 1..351: mol_type = other DNA
gaagtgcagctggtggagagcggaggaggattggtgcagcctggagagagcctgaggctgagttgtgccgcaagcggatttagtttttcctcctacgccatgtcctgggtgaggcagcctcctggaatgggaatcgaatgggtggccgagattgacagcggaggaggaagcaccgactacgctgttcccgtgaagggcaggtttaccatttcccgggacaacgttaagaacaccctgtatctgcagatgaacagcctgaagcctgaggacaccgccgtgttttattgcgccaagggcacccacgccgatttcggatcttggggacagggcacacaggtgaccgtgagttcc

SEQ ID NO: 104 - DNA, 357 bp, synthetic construct
  source 1..357: mol_type = other DNA
gaagtggacctggtggagagcggaggaggattggtgcaggcaggaggaagcctgaggctgagttgtgcccctagcggaaggacaagtagtctggatacaatggcatggttcaggcaggcccccggaaaggaaagggagttcgtggcttctaccaactggaatagaggaggagcatcttacgccgattccgtgaaggggaggttcaccatttccagggacgccactaagaacacagtctacctgcagatcaacagcctggagcctgaggacaccgccgtttactattgcgccgcctccaccaggggcatcgtttctgataactactggggccagggaacccaggtgaccgtttcttcc

SEQ ID NO: 105 - DNA, 360 bp, synthetic construct
  source 1..360: mol_type = other DNA
gaagtgcagctggtggaaagcggaggaggattggtgcagcctggaggaagtctgaggctgagttgtgcagcaagcaggggaacatttagcaggctggacatgggatggtttaggcaggcccctggaaaggaaagggagttcgtggctagttccacctggagcggaggaacagcatacgcaaccgacagtgtgaaaactcgttttacaatctctagggacagcgccaagacaacagtgtacctgcagatgaacagcctgaggcccgaggataccgctgtttattattgcgccgccggactgagcctggaacagcatacacctccctactggggccagggaacacaggttacagtgtcctct

SEQ ID NO: 106 - DNA, 366 bp, synthetic construct
  source 1..366: mol_type = other DNA
gaggttcagttggtggagtccggaggaggattggtgcaggctggaggaagtctgaggttgtcctgtgccgctagtggaaggacctttagtagggacgtgatggggtggtttaggcaggctcctggaaaggagagggagttcgtggctgtgattaactggactgactccacctactacctggattccgttaaggggaggttcaccatttcaagggagaacgctgagaacaccgtgtatctgcagatgaacagcctgaagcctgaagatactgctatttactactgcgccaccggcattgggggcctgtacaggaggccttccgcttacaagtattggggacagggcacacaggtcaccgttagtagc

SEQ ID NO: 107 - DNA, 384 bp, synthetic construct
  source 1..384: mol_type = other DNA
gctgttcagttggtggagtccgggggaggattggtgcaggctggaggatctctgaggttgtcttgcgccgcttctgggaggactttttcttcttattggttggggtggtttaggcaggtgcctggggaggaaagggagtttgtggctgctatttcttggagtggatctggcgtggtgactaattatgctgacagtgtgaagggccggtttactattagcagggacgacggaaacaacgccttgtacctgcagatgtccaatctgaaccccgaggacaccgctgtttattattgcgccgcccggctgggaacctattacaaggactacgcccggtccggggcctatgatacatggggacagggcacccaggtgaccgtttcttcc

SEQ ID NO: 108 - DNA, 351 bp, synthetic construct
  source 1..351: mol_type = other DNA
caggttcagctcgtcgagtcaggcggaggactcgttcagccaggagaaagcctcaggctgtcatgcgccgcttccggttttacattctcctcctacgccatgtcctgggtgaggcagccacctggaaagggtattgagtgggtggctgagattgacagcggaggagacaggacagactacgccgtgcctgtgaagggcaggtttaccatctccagagacaacgtggagaacacactctatctgcagatgaacagcttgaagcccgaagatactgctgtgttttactgcgccaaaggcacacacgccgatttcggaagctggggacagggaaccctggtgacagtgtcctcc

SEQ ID NO: 109 - DNA, 384 bp, synthetic construct
  source 1..384: mol_type = other DNA
gaggttgacttgatggagagcgggggaggattggtgcagcctggaggaagcctgagattgagctgtaccgctagtggagacacactggactactacgctatcgactggttcaggcaggcacctggaaaggagagggagggagttagctgcatttccagcaggggaggaaccaccaactacgctgacgctgtgaagggaagattcactatcagcagggacaacgccaagaacacagtgtacctgcagatgaacagcctgaggcccgaggataccgcagtttactactgcgccgccgagttcacaggatgggctgtgtgcaacttctccaggaagagcggagagtacgactactggggccagggaacacaggtgacagtgagtagc